(19) United States
(12) Patent Application Publication
(10) Pub. No.: US 2010/0137275 A1
(43) Pub. Date: Jun. 3, 2010

(54) TRIAMINOPYRIMIDINE DERIVATIVES AS INHIBITORS OF CDC25 PHOSPHATASE

(75) Inventors: Grégorie Prevost, Antony (FR); Anne-Marie Liberatore, Auffargis (FR); Dennis Bigg, Gif Sur Yvette (FR); Dominique Pons, Paris (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: IPSEN PHARMA S.A.S., Boulogne-Billancourt (FR)

(21) Appl. No.: 12/598,842

(22) PCT Filed: Apr. 30, 2008
(86) PCT No.: PCT/FR08/00620

§ 371 Date: Nov. 4, 2009

(30) Foreign Application Priority Data
May 4, 2007 (FR) 0703233

(51) Int. Cl. A61K 31/506; C07D 403/14; C07D 413/14; C07D 405/14; C07D 417/14; C07D 401/14; A61K 31/5377; C07D 239/42; A61K 31/505; A61K 31/498; A61P 35/00; A61P 35/02; A61P 33/00; A61P 31/00; A61P 29/00; A61P 25/28; A61P 17/14; A61P 37/06; A61P 37/08

(52) U.S. Cl. 514/210.2; 544/323; 514/275; 514/235.8; 544/122; 544/82; 514/232.2; 544/327; 514/256; 514/249

(57) ABSTRACT

The present invention relates to the novel triaminopyrimidine derivatives of formula (I) in which R1, R2, W, R3, R4, and R5 are variable groups. These products have a Cdc25-phosphatase-inhibiting activity. The invention also relates to a process for synthesizing these compounds and also to therapeutic compositions containing these products and to the use thereof as medicaments.

The present invention relates to novel triaminopyrimidine derivatives. These products have Cdc25 phosphatase-inhibiting activity. The invention also relates to a process for synthesising these compounds and to therapeutic compositions containing these products and to the use thereof as drugs.

Control of the transition between the different phases of the cell cycle during mitosis or meiosis is provided by a group of proteins, the enzymatic activity of which are associated with different phosphorylation states. These states are controlled by two large classes of enzyme: kinases and phosphatases.

In this way, the synchronisation of the different phases of the cell cycle enables the cell structure to be reorganised at each cycle in all living organisms (microorganisms, yeasts, vertebrates, plants). One group of kinases, the cyclin-dependent kinases (CDKs), has a major role in cell cycle control. The enzymatic activity of these various CDKs is controlled by two other families of enzymes that work in opposition (Jessus and Ozon, Prog. Cell Cycle Res. (1995), 1, 215-228). The first comprises kinases such as Weel and Mikl, which deactivate CDKs by phosphorylating certain amino acids (Den Haese et al., Mol. Biol. Cell (1995), 6, 371-385). The second comprises phosphatases such as Cdc25, which activate CDKs by dephosphorylating tyrosine and threonine residues of CDKs (Gould et al., Science (1990), 250, 1573-1576).

Phosphatases are classified into 3 groups: the serine/threonine phosphatases (PPases), the tyrosine phosphatases (PTPases) and the dual-specificity phosphatases (DSPases). These phosphatases play an important role in the regulation of many cell functions.

As far as the human Cdc25 phosphatases are concerned, 3 genes (cdc25-A, cdc25-B and cdc25-C) encode the Cdc25 proteins. In addition, variants originating from alternative splicing of the cdc25B gene have been identified: these splice variants are Cdc25B1, Cdc25B2 and Cdc25B3 (Baldin et al., Oncogene (1997), 14, 2485-2495).

The role of Cdc25 phosphatases in oncogenesis is now better understood and the mechanisms through which these phosphatases act are illustrated in the following references in particular: Galaktionov et al., Science (1995), 269, 1575-1577; Galaktionov et al., Nature (1996), 382, 511-517; and Mailand et al., Science (2000), 288, 1425-1429.

Overexpression of various forms of Cdc25 has now been reported in many human tumour series, for example:

Breast cancer: see Cangi et al., Abstract 2984, AACR meeting San Francisco, 2000);

Lymphomas: see Hernandez et al., Int. J. Cancer (2000), 89, 148-152 and Hernandez et al., Cancer Res. (1998), 58, 1762-1767;

Head and neck cancers: see Gasparotto et al., Cancer Res. (1997), 57, 2366-2368;

Pancreatic cancers: see Junchao Guo et al., Oncogene (2004), 23, 71-81.

Moreover, E. Sausville's group has reported a negative correlation between the level of Cdc25-B expression in a panel of 60 cell lines and their sensitivity to CDK inhibitors, suggesting that the presence of Cdc25 can provide resistance to certain antitumour agents and more particularly to CDK inhibitors (Hose et al., Proceedings of AACR, Abstract 3571, San Francisco, 2000).

In addition to other targets, compounds capable of inhibiting Cdc25 phosphatases are therefore being sought at present, particularly with a view to using them as anticancer agents.

Cdc25 phosphatases also have a role in neurodegenerative diseases (see Zhou et al., Cell Mol. Life. Sci. (1999), 56(9-10), 788-806; Ding et al., Am. J. Pathol. (2000), 157(6), 1983-90; Vincent et al., Neuroscience (2001), 105(3), 639-50) and the use of compounds with inhibitory activity against these phosphatases can therefore also be envisaged in the treatment of these diseases.

Another problem addressed by the invention is the search for drugs for the prevention or treatment of organ graft rejection or for the treatment of autoimmune diseases. These disorders and/or diseases involve inappropriate activation of lymphocytes and monocytes/macrophages. However current immunosuppressant agents have side effects that could be reduced or modified by products that specifically target the signalling pathways in haemopoietic cells that initiate and maintain inflammation.

The triaminopyrimidine derivatives hereinafter defined are novel Cdc25 phosphatase inhibitors. They could be used as drugs, particularly in the treatment and/or prevention of the following diseases or disorders:
inhibition of tumour proliferation, alone or in combination with other treatments;
cancers:
inhibition of the proliferation of normal cells, alone or in combination with other treatments;
neurodegenerative diseases;
prevention of spontaneous alopecia;
prevention of alopecia induced by exogenous products;
prevention of radiation-induced alopecia;
prevention of spontaneous or induced apoptosis of normal cells;
prevention of meiosis and/or fertilisation;
prevention of oocyte maturation;
any diseases and/or any disorders corresponding to uses reported for CDK inhibitors, and particularly noncancerous proliferative diseases (for example: angiogenesis, psoriasis or restenosis), cancerous proliferative diseases, parasitic diseases (protozoan proliferation), viral infections, neurodegenerative diseases, myopathies; and/or
any disease and/or any disorder corresponding to clinical applications of vitamin K and its derivatives.

In addition, on account of their Cdc25 phosphatase-inhibiting properties, the compounds of the present invention could also be used to inhibit or prevent the proliferation of microorganisms, and particularly yeasts. One of the advantages of these compounds is their low toxicity for healthy cells.

The present invention relates to a compound having the general formula (I)

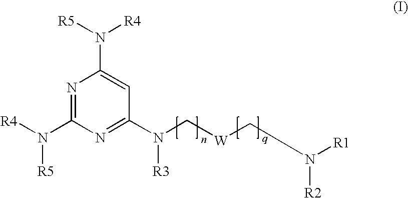

in a racemic form, an enantiomeric form or any combination thereof, wherein:
R1 represents a hydrogen atom, an alkyl group, a —C(═O)—NHR8, —C(═S)—NHR8, —C(═S)—NH—C(═O)—R8, —C(═N—CN)—NHR8, —C(═O)—R9 or —SO₂—R10 group;
R2 represents a hydrogen atom, a C₁-C₃ linear or branched alkyl group;
W represents —NR6-, —CR6R7-, an oxygen atom or a sulfur atom;
R6 and R7 represent independently a hydrogen atom or an alkyl group;
n or q are integers from 2 to 6 inclusive;
R3 represents a hydrogen atom or an alkyl group;
R4 and R5 represent independently a hydrogen atom, an alkyl group, an aminoalkyl, alkylaminoalkyl or dialkylaminoalkyl group; or alternatively R4 and R5 together with the nitrogen atom to which they are attached form a heterocycloalkyl;
R8 represents a hydrogen atom or one of the following groups:
   alkyl,
   cycloalkyl,
   heterocycloalkylalkyl,
   heteroaryl optionally substituted by one or more identical or different groups selected from: alkyl, heterocycloalkyl, halo and aryloxy optionally substituted by one or more identical or different halogens,
   heteroarylalkyl,
   aryl optionally substituted by one or more identical or different groups selected from: alkyl; alkoxy; alkylthio; dialkylamino; halo; haloalkyl; haloalkyloxy; cyano; nitro; heteroaryl; heteroarylthio optionally substituted by one or more identical or different groups selected from halo, haloalkyl; aryloxy optionally substituted by one to or more nitro groups; arylsulfonyl optionally substituted by one or more identical or different halogens; or a —SO₂NR15R16 group;
   an arylalkyl group optionally substituted by one or more identical or different halogens, or;
   a group having the formula:

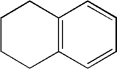

R9 represents one of the following groups:
   aryl optionally substituted by one or more arylcarbonyl groups,
   aryloxyalkyl optionally substituted by a C₁-C₃ alkyl group,
   heteroaryl;
   heterocycloalkyl optionally substituted by a heteroaryl, itself optionally substituted by a haloalkyl group;
R10 represents an aryl group optionally substituted by one or more identical or different groups selected from haloalkyl, nitro;
R15 and R16 represent independently a heteroaryl group optionally substituted by one or more identical or different C₁-C₃ alkyls, a C₁-C₃ alkyl group, an aryl group or a hydrogen atom; or alternatively R15 and R16 can together form a heterocycloalkyl including the nitrogen atom;
or a pharmaceutically acceptable salt thereof.

The terminology used hereinafter for the nomenclature of the compounds and the examples is the English IUPAC terminology.

When no further details are given, alkyl is understood to mean a linear or branched alkyl group containing 1 to 6 carbon atoms, preferably 1 to 4 carbon atoms, such as methyl, ethyl, propyl, isopropyl, butyl, isobutyl, tert-butyl, pentyl or hexyl.

Alkylamino or dialkylamino is understood in the present invention to mean an amino group substituted by one or two alkyl groups as hereinbefore defined, such as methylamino, dimethylamino, methylethylamino, ethylamino or diethylamino.

Aminoalkyl, alkylaminoalkyl or dialkylaminoalkyl are understood to mean an alkyl group as hereinbefore defined, substituted by an amino group or by an alkylamino or dialkylamino group as hereinbefore defined, such as dimethylaminoethyl or diethylaminoethyl.

Alkoxy is understood in the present invention to mean an —O-alkyl group wherein the alkyl moiety is as hereinbefore defined, such as the methoxy or ethoxy group.

Alkylthio is understood in the present invention to mean an —S-alkyl group wherein the alkyl group is as hereinbefore defined, such as the methylthio or ethylthio group.

Haloalkyl is understood to mean an alkyl group as hereinbefore defined, substituted by one or more identical or different halogen atoms, such as trifluoromethyl or pentafluoroethyl.

Haloalkyloxy is understood to mean an —O-(haloalkyl) group wherein the haloalkyl group is as hereinbefore defined, such as the trifluoromethoxy group.

When no further details are given, cycloalkyl is understood to mean a saturated 3- to 6-membered cyclic carbon group, such as cyclopropyl, cyclobutyl, cyclopentyl or cyclohexyl and preferably cyclopentyl and cyclohexyl.

Heterocycloalkyl (or heterocyl [translator's note: possibly a typo for heterocycle]) is understood in the present invention to mean a 3- to 6-membered ring including one or more identical or different heteroatoms selected from O, N and S, such as an azeridinyl [translator's note: possibly aziridinyl], azetidinyl, pyrrolidinyl, piperidinyl, morpholinyl or tetrahydrofuran group.

Heterocycloalkylalkyl is understood to mean an alkyl group substituted by a heterocycloalkyl as hereinbefore defined, such as the tetrahydrofuryl-methyl group.

Aryl (or aromatic carbocycle) is understood to mean an unsaturated carbocyclic system including at least one aromatic ring and preferably one moiety selected from phenyl, naphthyl and fluorenyl.

Aryloxy is understood to mean an —O-aryl group wherein the aryl group is as hereinbefore defined such as the phenoxy group.

Arylalkyl is understood to mean an alkyl group as hereinbefore defined, substituted by an aryl group as hereinbefore defined, such as the benzyl group or the phenethyl group.

Arylcarbonyl is understood to mean a carbonyl group substituted by an aryl group as hereinbefore defined, such as the phenylcarbonyl group.

Aryloxyalkyl is understood in the present invention to mean an alkyl group substituted by aryloxy as hereinbefore defined, such as phenoxymethyl, phenoxyethyl.

Arylsulfonyl is understood to mean an SO₂-aryl group wherein the aryl moiety is as hereinabove defined, such as the phenylsulfonyl group.

Heteroaryl is understood in the present invention to mean an unsaturated aromatic ring containing one or more identical or different heteroatoms selected from N, O and S such as furyl, thienyl isoxazolyl, benzothiadiazolyl, pyridinyl, oxazolyl, pyrazolyl, pyrimidinyl or quinoxalyl.

Heteroarylalkyl is understood to mean an alkyl group substituted by a heteroaryl as hereinabove defined, such as the furylmethyl group.

Heteroarylthio is understood in the present invention to mean an —S-heteroaryl group wherein the heteroaryl moiety is as hereinbefore defined such as the pyridylthio group.

Salt of a compound is understood to mean acid addition salts thereof with an organic or inorganic acid or, where appropriate, base addition salts, and in particular pharmaceutically acceptable salts of said compound.

Pharmaceutically acceptable salt is understood in particular to mean acid addition salts with inorganic acids such as hydrochloride, hydrobromide, hydroiodide, sulfate, phosphate, diphosphate and nitrate or with organic acids such as acetate, maleate, fumarate, tartrate, succinate, citrate, lactate, methanesulfonate, p-toluenesulfonate, pamoate and stearate. Salts formed from bases such as sodium hydroxide or potassium hydroxide also fall under the scope of the present invention, when they are usable. For other examples of pharmaceutically acceptable salts, “Salt selection for basic drugs”, Int. J. Pharm. (1986), 33, 201-217, may be referred to.

In some cases, the compounds of the present invention may contain asymmetric carbon atoms. The compounds of the present invention therefore have two possible enantiomeric forms, i.e. the “R” and “S” configurations. The present invention includes both enantiomeric forms and any combinations thereof, including “RS” racemic mixtures. For the sake of simplicity, when no specific configuration is indicated in the structural formulae, it should be taken to mean that both enantiomeric forms and mixtures thereof are represented.

The invention also relates to a compound having the general formula (I) characterised in that R4 and R5 independently represent a hydrogen atom, an alkyl, aminoalkyl, alkylaminoalkyl or dialkylaminoalkyl group.

The invention also relates to a compound having the general formula (I) characterised in that R4 and R5 together with the nitrogen atom to which they are attached form a heterocycloalkyl.

The invention preferably relates to a compound having the general formula (I) characterised in that W represents —CR6R7-, and more preferably wherein W represents —CR6R7- and R1 represents —C(═S)—NHR8, —C(═S)—NH—C(═O)—R8 or —C(═N—CN)—NHR8.

The invention particularly relates to a compound having the general formula (I) wherein

R1 represents a —C(═S)—NHR8 group;
R2 represents a hydrogen atom;
W represents —CR6R7-;
R6 and R7 represent independently a hydrogen atom or an alkyl group;
R3 represents a hydrogen atom;
R4 and R5 together with the nitrogen atom to which they are attached form a heterocycloalkyl that includes only one nitrogen atom; and
R8 represents an aryl group optionally substituted by one or more identical or different groups selected from alkyl, alkoxy; alkylthio; halo; haloalkyl; cyano; nitro; heteroarylthio optionally substituted by one or more identical or different groups selected from halo, haloalkyl; aryloxy optionally substituted by one or more nitro groups; and preferably when the term heterocycloalkyl denotes a pyrrolidine or a piperidine; the term aryl of aryl and aryloxy groups is the phenyl group; and the heteroarylthio group is the pyridinylthio group.

The invention also preferably concerns a compound having the general formula (I) characterised in that W represents —NR6- or an oxygen atom, and more preferably W represents —NR6- or an oxygen atom and R1 represents a —C(═O)—NHR8, —C(═S)—NHR8, —C(═S)—NH—C(═O)—R8, —C(═N—CN)—NHR8, —C(═O)—R9 or —SO₂—R10 group. In these cases, the invention particularly concerns a compound wherein R2 represents a hydrogen atom, R4 and R5 together with the nitrogen atom to which they are attached form a heterocycloalkyl consisting of only carbon, nitrogen, and optionally oxygen atoms, and preferably a heterocycloalkyl including only one nitrogen atom.

The invention also preferably relates to a compound having the general formula (I) characterised in that:

R1 represents either a —C(═O)—NHR8, —C(═S)—NHR8 group;
R2 represents a hydrogen atom;
W represents —NR6- or the oxygen atom;
R6 represents an alkyl group;
R3 represents a hydrogen atom;
R4 and R5 represent independently a hydrogen atom, an alkyl group; or alternatively R4 and R5 together with the nitrogen atom to which they are attached form a heterocycloalkyl including only one nitrogen atom;
R8 represents an aryl group optionally substituted by one or more identical or different groups selected from alkyl, alkoxy; alkylthio; halo; haloalkyl; cyano; nitro; heteroarylthio optionally substituted by one or more identical or different groups selected from halo, haloalkyl; aryloxy optionally substituted by one or more nitro groups; or a —SO₂NR15R16 group;
R15 and R16 may together form a heterocycloalkyl group including the nitrogen atom, or R15 and R16 independently represent a heteroaryl group optionally substituted by one or more identical or different C₁-C₃ alkyls, a C₁-C₃ alkyl group, an aryl group or a hydrogen atom; and preferably when the term heterocycloalkyl denotes a pyrrolidine or a piperidine; the term aryl of aryl and aryloxy groups is the phenyl group; and the term heteroaryl of heteroaryl and heteroarylthio groups represents a pyridine or a pyrimidine.

In the compounds of the present invention of general formula (I),
the term aryl of aryl, aryloxy, arylsulfonyl, arylalkyl, aryloxyalkyl and arylcarbonyl preferably represents the phenyl, naphthyl or fluororenyl [translator's note: possible fluorenyl] group, and/or
the term heteroaryl of heteroaryl, heteroarylalkyl and heteroarylthio groups preferably represents the furyl, thienyl isoxazolyl, benzothiadiazolyl, pyridinyl, oxazolyl, pyrazolyl, pyrimidinyl or quinoxalyl group; and/or
the term cycloalkyl preferably represents cyclopentyl or cyclohexyl; and/or
the term heterocycloalkyl of heterocycloalkyl and heterocycloalkylalkyl groups preferably represents the tetrahydrofuryl, azetidinyl, pyrrolidinyl, morpholinyl or piperidyl group.

The present invention also relates to compounds having the general formula (I)

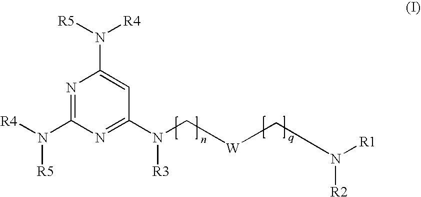

in a racemic form, an enantiomeric form or any combination thereof, wherein:
W represents independently NR6, CR6R7, an oxygen atom or a sulfur atom, it being understood that R6 and R7 independently represent a hydrogen atom or a linear or C₁-C₆ branched alkyl group;
R3 represents a hydrogen atom or a linear or branched C₁-C₆ alkyl group;
R2 represents a hydrogen atom, a linear or branched C₁-C₃ alkyl group;
or R4 and R5 together form a heterocyclic ring including the nitrogen atom;
or R4 and R5 independently represent a hydrogen atom, a linear or branched C₁-C₆ alkyl group, a phenyl group, a alkylaminoalkyl group or a —(CH₂)₂—N(CH₃)₂ group;
n or q are integers [sic] from 2 to 6 inclusive;
R1 represents either a hydrogen atom, a —C(═O)—NHR8, —C(═S)—NHR8, —C(═S)—NH—C(═O)—R8, —C(═N—CN)—NHR8, —C(═O)—R9 or —SO₂—R10 group;
R8 represents either a hydrogen atom, a linear or branched C₁-C₆ alkyl group, a thiophene group, a naphthyl group, a tetrahydronaphthyl group, a cyclopentyl group, a benzothiadiazole group, an isoxazole group optionally substituted by 1 or 2 C₁-C₂ alkyl groups, a methylfuryl group, a tetrahydrofuryl group, a benzyl group optionally substituted by a halogen atom, or alternatively a pyridine group optionally substituted by a phenoxy group, by a halogen atom, by a halogenophenoxy group or by a morpholino group;
or alternatively
R8 represents a

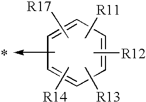

group wherein R11, R12, R13, R14 or R17 independently represent a hydrogen atom, a halogen atom, a —CN, —NO₂, —OCF₃, —CF₃ group, an alkylthio group, an alkylamino group, an oxazole group, a pyrazole group, an alkoxy group, a phenoxy group optionally substituted by a —NO₂ group, a linear or branched C₁-C₆ alkyl group, a thiopyridine group optionally substituted by a halogen atom and by a —CF₃ group, a arylsulfone group optionally substituted by a halogen atom,
or alternatively R11, R12, R13, R14 or R17 independently represent an —SO₂—NR15R16 group, it being understood that R15 and R16 may form together a heterocyclic ring including the nitrogen atom; or
R15 or R16 independently represent a dimethylpyrimidine group, a C₁-C₃ alkyl group, a phenyl group or a hydrogen atom;
R9 represents a

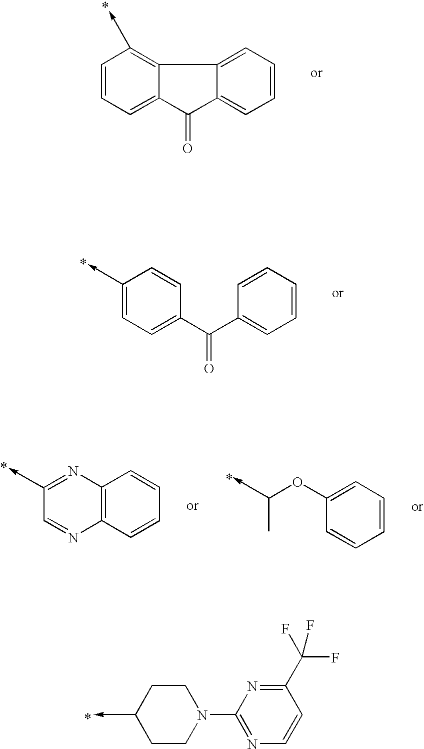

group;
R10 represents a

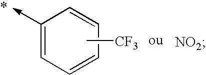

group, it being understood that each time it is used [symbol] means the point of attachment to the general formula (I);
or a pharmaceutically acceptable salt thereof.

Preferably, the present invention relates to compounds having the general formula (I)

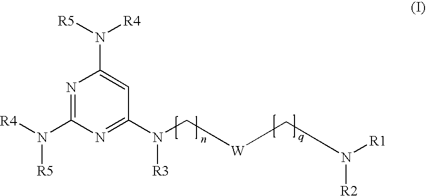

in a racemic form, an enantiomeric form or any combination thereof, wherein:
W independently represents NR6 where R6 represents a hydrogen atom or a linear or branched C₁-C₆ alkyl group;
R3 represents a hydrogen atom, a linear or branched C₁-C₆ alkyl group;
R2 represents independently a hydrogen atom, a linear or branched C₁-C₃ alkyl group;
R4 and R5 together form a heterocyclic ring including the nitrogen atom; n or q are integers [sic] from 2 to 6 inclusive;
R1 represents either a hydrogen atom, a —C(═O)—NHR₈ group, or a —C(═S)—NHR₈ group;
R8 represents either a hydrogen atom, a linear or branched C₁-C₄ alkyl group, a thiophene group, a methylphenoxy group, a naphthyl group, a dihydronaphthyl group, a cyclopentyl group, a benzothiadiazole group, an isoxazole group optionally substituted by 1 or 2 methyl groups, a pyrazole group, a methylfuryl group, a methyldihydrofuryl group, a benzyl group optionally substituted by a fluorine atom, or a pyridine group optionally substituted by a phenoxy group, by a halogen atom, by a fluorophenoxy group or by a morpholino group;
or alternatively
R8 represents a

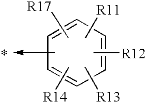

group wherein R11, R12, R13, R14 or R17 independently represent a hydrogen atom, a halogen atom, a —CN, —NO₂, —OCF₃, —CF₃, —S—CH₃ group, a dimethyl amine group, an oxazole group, a methoxy group, a phenoxy group optionally substituted by a —NO₂ group, a linear or branched C₁-C₆ alkyl group, thiopyridine optionally substituted by halogen atoms or —CF₃, a arylsulfone group optionally substituted by a halogen atom, or alternatively R11, R12, R13, R14 or R17 independently represent an —SO2-NR15R16 group, it being understood that R15 and R16 may together form a heterocyclic ring including the nitrogen atom;
or a pharmaceutically acceptable salt thereof.

Preferably, the compound of the invention has R4 and R5 groups that together form a heterocyclic ring including the nitrogen atom, and more particularly that form a pyrrolidine group.

Preferably, the compound of the invention has an R3 group that represents a hydrogen atom.

Preferably, the compound of the invention is such that n or q are integers equal to 2 or 3, and more particularly n and q are equal to 2.

Preferably, the compound of the invention has a W group that represents an NR6 group, and more particularly that represents an NR6 group with R6 being a linear alkyl group.

Preferably, the compound of the invention has an R2 group that represents a hydrogen atom and an R1 group that represents a —C(═O)—NHR8 group or a —C(═S)—NHR8 group.

More preferably still, the compound of the invention has an R2 group that represents a hydrogen atom and an R1 group that represents a —C(═S)—NHR8 group.

Preferably, the compound of the invention has an R2 group that represents a hydrogen atom and R1 that represents either a —C(═O)—NHR8 group, or a —C(═S)—NHR8 group, with R8 being a

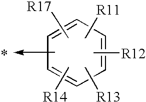

group wherein R11, R12, R13, R14 or R17 independently represent a hydrogen atom, a halogen atom, a —CN, —NO₂, —CF₃ group, an alkoxy group or a phenoxy group.

More particularly the invention also relates to a compound having the general formula (I) characterised in that it is selected from:
N-{2-[(2,6-dipyrrolidin-1-ylpyrimidin-4-yl)amino]ethyl}-N-methylethane-1,2-diamine;
N-(3-aminopropyl)-N′-(2,6-dipyrrolidin-1-ylpyrimidin-4-yl)-N-methylpropane-1,3-diamine;
N-(2,6-dipyrrolidin-1-ylpyrimidin-4-yl)-N,N′-dimethyl-N′-[3-(methylamino)propyl]propane-1,3-diamine;
N-(2,6-dipyrrolidin-1-ylpyrimidin-4-yl)pentane-1,5-diamine;
N′-(2,6-dipyrrolidin-1-ylpyrimidin-4-yl)-N,N-dimethylpentane-1,5-diamine;
N′-(2,6-dipyrrolidin-1-ylpyrimidin-4-yl)-N,N-diethylpentane-1,5-diamine;
N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea;
N-benzyl-N′-{2-[{2-[(2,6-dipyrrolidin-1-ylpyrimidin-4-yl)amino]ethyl}(methyl)amino]ethyl}urea;
N-(tert-butyl)-N′-{2-[{2-[(2,6-dipyrrolidin-1-ylpyrimidin-4-yl)amino]ethyl}(methyl)amino]ethyl}urea;
N-{2-[{2-[(2,6-dipyrrolidin-1-ylpyrimidin-4-yl)amino]ethyl}(methyl)amino]ethyl}-N′-2-thienylurea;
N-{2-[{2-[(2,6-dipyrrolidin-1-ylpyrimidin-4-yl)amino]ethyl}(methyl)amino]ethyl}-N′-[(2R)-1,2,3,4-tetrahydronaphthalen-2-yl]urea;
N-cyclopentyl-N′-{2-[{2-[(2,6-dipyrrolidin-1-ylpyrimidin-4-yl)amino]ethyl}(methyl)amino]ethyl}urea;
N-(3,5-dimethylisoxazol-4-yl)-N′-{2-[{2-[(2,6-dipyrrolidin-1-ylpyrimidin-4-yl)amino]ethyl}(methyl)amino]ethyl}urea;
N-{2-[{2-[(2,6-dipyrrolidin-1-ylpyrimidin-4-yl)amino]ethyl}(methyl)amino]ethyl}-N′-(2-furylmethyl)urea;
N-2,1,3-benzothiadiazol-4-yl-N′-{2-[{2-[(2,6-dipyrrolidin-1-ylpyrimidin-4-yl)amino]ethyl}(methyl)amino]ethyl}urea;
N-(4-chlorophenyl)-N′-{2-[{2-[(2,6-dipyrrolidin-1-ylpyrimidin-4-yl)amino]ethyl}(methyl)amino]ethyl}urea;
N-(3,4-dichlorophenyl)-N′-{2-[{2-[(2,6-dipyrrolidin-1-ylpyrimidin-4-yl)amino]ethyl}(methyl)amino]ethyl}urea;
N-{2-[{2-[(2,6-dipyrrolidin-1-ylpyrimidin-4-yl)amino]ethyl}(methyl)amino]ethyl}-N′-ethylurea;
N-(4-chlorophenyl)-N′-{5-[(2,6-dipyrrolidin-1-ylpyrimidin-4-yl)amino]pentyl}urea;
N-[4-chloro-3-(trifluoromethyl)phenyl]-N′-(2-{2-[(2,6-dipyrrolidin-1-ylpyrimidin-4-yl)amino]ethoxy}ethyl)urea;
N-[4-chloro-3-(trifluoromethyl)phenyl]-N′-{3-[{3-[(2,6-dipyrrolidin-1-ylpyrimidin-4-yl)amino]propyl}(methyl)amino]propyl}urea;
N-(4-chlorophenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-[4-(trifluoromethoxy)phenyl]thiourea;
N-{2-[{2-[(2,6-dipyrrolidin-1-ylpyrimidin-4-yl)amino]ethyl}(methyl)amino]ethyl}-N′-(4-fluorophenyl)thiourea;
N-{2-[{2-[(2,6-dipyrrolidin-1-ylpyrimidin-4-yl)amino]ethyl}(methyl)amino]ethyl}-N′-[4-(trifluoromethyl)phenyl]thiourea;
N-{2-[{2-[(2,6-dipyrrolidin-1-ylpyrimidin-4-yl)amino]ethyl}(methyl)amino]ethyl}-N′-pyridin-3-ylthiourea;
N-{2-[{2-[(2,6-dipyrrolidin-1-ylpyrimidin-4-yl)amino]ethyl}(methyl)amino]ethyl}-N′-[4-(piperidin-1-ylsulfonyl)phenyl]thiourea;
N-{2-[{2-[(2,6-dipyrrolidin-1-ylpyrimidin-4-yl)amino]ethyl}(methyl)amino]ethyl}-N′-ethylthiourea;
N-{2-[{2-[(2,6-dipyrrolidin-1-ylpyrimidin-4-yl)amino]ethyl}(methyl)amino]ethyl}-N′-(2-furylmethyl)thiourea;
N-{2-[{2-[(2,6-dipyrrolidin-1-ylpyrimidin-4-yl)amino]ethyl}(methyl)amino]ethyl}-N′-(6-phenoxypyridin-3-yl)thiourea;
N-{2-[{2-[(2,6-dipyrrolidin-1-ylpyrimidin-4-yl)amino]ethyl}(methyl)amino]ethyl}-N′-(tetrahydrofuran-2-ylmethyl)thiourea;
N-{2-[{2-[(2,6-dipyrrolidin-1-ylpyrimidin-4-yl)amino]ethyl}(methyl)amino]ethyl}-N′-(6-morpholin-4-ylpyridin-3-yl)thiourea;
N-{2-[{2-[(2,6-dipyrrolidin-1-ylpyrimidin-4-yl)amino]ethyl}(methyl)amino]ethyl}-N′-[4-(1,3-oxazol-5-yl)phenyl]thiourea;
N-{2-[{2-[(2,6-dipyrrolidin-1-ylpyrimidin-4-yl)amino]ethyl}(methyl)amino]ethyl}-N′-(pentafluorophenyl)thiourea;
N-{2-[{2-[(2,6-dipyrrolidin-1-ylpyrimidin-4-yl)amino]ethyl}(methyl)amino]ethyl}-N′-(4-methoxyphenyl)thiourea;
N-{2-[{2-[(2,6-dipyrrolidin-1-ylpyrimidin-4-yl)amino]ethyl}(methyl)amino]ethyl}-N′-(4-phenoxyphenyl)thiourea;
N-{2-[{2-[(2,6-dipyrrolidin-1-ylpyrimidin-4-yl)amino]ethyl}(methyl)amino]ethyl}-N′-1-naphthylthiourea;
N-{2-[{2-[(2,6-dipyrrolidin-1-ylpyrimidin-4-yl)amino]ethyl}(methyl)amino]ethyl}-N′-(3,4,5-trimethoxyphenyl)thiourea;
N-{2-[{2-[(2,6-dipyrrolidin-1-ylpyrimidin-4-yl)amino]ethyl}(methyl)amino]ethyl}-N′-(3-fluorophenyl)thiourea;
N-(2,4-difluorophenyl)-N′-{2-[{2-[(2,6-dipyrrolidin-1-ylpyrimidin-4-yl)amino]ethyl}(methyl)amino]ethyl}thiourea;
N-(3,5-difluorophenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-(2-fluorophenyl)thiourea;
N-{2-[{2-[(2,6-dipyrrolidin-1-ylpyrimidin-4-yl)amino]ethyl}(methyl)amino]ethyl}-N′-(4-nitrophenyl)thiourea;
N-(4-tert-butylphenyl)-N′-{2-[{2-[(2,6-dipyrrolidin-1-ylpyrimidin-4-yl)amino]ethyl}(methyl)amino]ethyl}thiourea;
N-{-2-[{2-[(2,6-dipyrrolidin-1-ylpyrimidin-4-yl)amino]ethyl}(methyl)amino]ethyl}-N′-[4-(4-nitrophenoxy)phenyl]thiourea;
N-{2-[{2-[(2,6-dipyrrolidin-1-ylpyrimidin-4-yl)amino]ethyl}(methyl)amino]ethyl}-N′-(4-fluorobenzyl)thiourea;
N-[2-(2,4-difluorophenoxy)pyridin-3-yl]-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-[4-(1H-pyrazol-1-yl)phenyl]thiourea;
N-{2-[{2-[(2,6-dipyrrolidin-1-ylpyrimidin-4-yl)amino]ethyl}(methyl)amino]ethyl}-N′-(3-nitrophenyl)thiourea;
N-(4,6-dimethylpyrimidin-2-yl)-4-{[({2-[{2-[(2,6-dipyrrolidin-1-ylpyrimidin-4-yl)amino]ethyl}(methyl)amino]ethyl}amino)carbonothioyl]amino}benzenesulfonamide;
N-{2-[{2-[(2,6-dipyrrolidin-1-ylpyrimidin-4-yl)amino]ethyl}(methyl)amino]ethyl}-N′-[4-(methylthio)phenyl]thiourea;
N-(4-{[3-chloro-5-(trifluoromethyl)pyridin-2-yl]thio}phenyl)-N′-{2-[{2-[(2,6-dipyrrolidin-1-ylpyrimidin-4-yl)amino]ethyl}(methyl)amino]ethyl}thiourea;
N-(6-chloropyridin-3-yl)-N′-{2-[{2-[(2,6-dipyrrolidin-1-ylpyrimidin-4-yl)amino]ethyl}(methyl)amino]ethyl}thiourea;
N-[4-chloro-3-(trifluoromethyl)phenyl]-N′-{2-[{2-[(2,6-dipyrrolidin-1-ylpyrimidin-4-yl)amino]ethyl}(methyl)amino]ethyl}thiourea;
N-(3,4-dichlorophenyl)-N′-{2-[{2-[(2,6-dipyrrolidin-1-ylpyrimidin-4-yl)amino]ethyl}(methyl)amino]ethyl}thiourea;
N-(4-chloro-3-fluorophenyl)-N′-{2-[{2-[(2,6-dipyrrolidin-1-ylpyrimidin-4-yl)amino]ethyl}(methyl)amino]ethyl}thiourea;
N-[4-chloro-3-(trifluoromethyl)phenyl]-N′-{5-[(2,6-dipyrrolidin-1-ylpyrimidin-4-yl)amino]pentyl}thiourea;
N-(4-chlorophenyl)-N′-{5-[(2,6-dipyrrolidin-1-ylpyrimidin-4-yl)amino]pentyl}thiourea;
4-{[({2-[{2-[(2,6-dipyrrolidin-1-ylpyrimidin-4-yl)amino]ethyl}(methyl)amino]ethyl}amino)carbonothioyl]amino}-N-methylbenzenesulfonamide;
N-{4-[(4-bromophenyl)sulfonyl]phenyl}-N′-{2-[{2-[(2,6-dipyrrolidin-1-ylpyrimidin-4-yl)amino]ethyl}(methyl)amino]ethyl}thiourea;
4-{[({2-[{2-[(2,6-dipyrrolidin-1-ylpyrimidin-4-yl)amino]ethyl}(methyl)amino]ethyl}amino)carbonothioyl]amino}benzenesulfonamide;
4-{[({2-[{2-[(2,6-dipyrrolidin-1-ylpyrimidin-4-yl)amino]ethyl}(methyl)amino]ethyl}amino)carbonothioyl]amino}N-phenylbenzenesulfonamide;
N˜6˜-{2-[(2-aminoethyl)(methyl)amino]ethyl}-N˜2˜,N˜4˜-bis[2-(dimethylamino)ethyl]-N˜2˜,N˜4˜-dimethylpyrimidine-2,4,6-triamine
N-{2-[[2-({2,6-bis[[2-(dimethylamino)ethyl](methyl)amino]pyrimidin-4-yl}amino)ethyl](methyl)amino]ethyl}-N′-(4-chlorophenyl)thiourea;
N-{2-[(2,6-dimorpholin-4-ylpyrimidin-4-yl)amino]ethyl}-N-methylethane-1,2-diamine
N-(4-chlorophenyl)-N′-{2-[{2-[(2,6-dimorpholin-4-ylpyrimidin-4-yl)amino]ethyl}(methyl)amino]ethyl}thiourea;
N-(3,4-difluorophenyl)-N′-{2-[{2-[(2,6-dipyrrolidin-1-ylpyrimidin-4-yl)amino]ethyl}(methyl)amino]ethyl}thiourea;
N-{2-[(2,6-dipiperidin-1-ylpyrimidin-4-yl)amino]ethyl}-N-methylethane-1,2-diamine;
N-(4-chlorophenyl)-N′-{2-[{2-[(2,6-dipiperidin-1-ylpyrimidin-4-yl)amino]ethyl}(methyl)amino]ethyl}thiourea;
N˜6˜-{2-[(2-amino ethyl)(methyl)amino]ethyl}-N˜2˜,N˜2˜,N˜4˜,N˜4˜-tetraethylpyrimidine-2,4,6-triamine;
N-{2-[(2-{[2,6-bis(diethylamino)pyrimidin-4-yl]amino}ethyl)(methyl)amino]ethyl}-N′-(4-chlorophenyl)thiourea;
N˜6˜-{2-[(2-amino ethyl)(methyl)amino]ethyl}-N˜2˜,N˜2˜,N˜4˜,N˜4˜-tetramethylpyrimidine-2,4,6-triamine;
N-{2-[(2-{[2,6-bis(dimethylamino)pyrimidin-4-yl]amino}ethyl)(methyl)amino]ethyl}-N′-(4-chlorophenyl)thiourea;
N-{2-[(2,6-diazetidin-1-ylpyrimidin-4-yl)amino]ethyl}-N-methylethane-1,2-diamine;
N-(4-chlorophenyl)-N′-{2-[{2-[(2,6-diazetidin-1-ylpyrimidin-4-yl)amino]ethyl}(methyl)amino]ethyl}thiourea;
N-[4-(dimethylamino)phenyl]-N′-{2-[{2-[(2,6-dipyrrolidin-1-ylpyrimidin-4-yl)amino]ethyl}(methyl)amino]ethyl}thiourea;
N-(4-cyanophenyl)-N′-{2-[{2-[(2,6-dipyrrolidin-1-ylpyrimidin-4-yl)amino]ethyl}(methyl)amino]ethyl}thiourea;
N-4˜-{2-[(2-aminoethyl)(methyl)amino]ethyl}-N˜2˜,N˜6˜-diethylpyrimidine-2,4,6-triamine
N-{2-[(2-{[2,6-bis(ethylamino)pyrimidin-4-yl]amino}ethyl)(methyl)amino]ethyl}-N′-(4-chlorophenyl)thiourea;
N-[({2-[{2-[(2,6-dipyrrolidin-1-ylpyrimidin-4-yl)amino]ethyl}(methyl)amino]ethyl}amino)carbonothioyl]-4-methoxybenzamide;
N-(4-chlorophenyl)-N″-cyano-N′-{2-[{2-[(2,6-dipyrrolidin-1-ylpyrimidin-4-yl)amino]ethyl}(methyl)amino]ethyl}guanidine;
N-{2-[{2-[(2,6-dipyrrolidin-1-ylpyrimidin-4-yl)amino]ethyl}(methyl)amino]ethyl}quinoxaline-2-carboxamide
4-benzoyl-N-{2-[{2-[(2,6-dipyrrolidin-1-ylpyrimidin-4-yl)amino]ethyl}(methyl)amino]ethyl}benzamide;
N-{2-[{2-[(2,6-dipyrrolidin-1-ylpyrimidin-4-yl)amino]ethyl}(methyl)amino]ethyl}-2-phenoxypropanamide;
N-{2-[{2-[(2,6-dipyrrolidin-1-ylpyrimidin-4-yl)amino]ethyl}(methyl)amino]ethyl}-9-oxo-9H-fluorene-4-carboxamide;
N-{2-[{2-[(2,6-dipyrrolidin-1-ylpyrimidin-4-yl)amino]ethyl}(methyl)amino]ethyl}-1-[4-(trifluoromethyl)pyrimidin-2-yl]piperidine-4-carboxamide;
N-{2-[{2-[(2,6-dipyrrolidin-1-ylpyrimidin-4-yl)amino]ethyl}(methyl)amino]ethyl}-4-nitrobenzenesulfonamide;
N-{2-[{2-[(2,6-dipyrrolidin-1-ylpyrimidin-4-yl)amino]ethyl}(methyl)amino]ethyl}-3-(trifluoromethyl)benzenesulfonamide;
N-{2-[{2-[(2,6-dipyrrolidin-1-ylpyrimidin-4-yl)amino]ethyl}(methyl)amino]ethyl}-4-(trifluoromethyl)benzenesulfonamide;
   or a pharmaceutically acceptable salt thereof,
   and more preferably, it is selected from:
N-[4-chloro-3-(trifluoromethyl)phenyl]-N′-(2-{2-[(2,6-dipyrrolidin-1-ylpyrimidin-4-yl)amino]ethoxy}ethyl)urea;
N-(4-chlorophenyl)-N′-{5-[(2,6-dipyrrolidin-1-ylpyrimidin-4-yl)amino]pentyl}urea;
N-[4-chloro-3-(trifluoromethyl)phenyl]-N′-(2-{2-[(2,6-dipyrrolidin-1-ylpyrimidin-4-yl)amino]ethoxy}ethyl)urea;
N-[4-chloro-3-(trifluoromethyl)phenyl]-N′-{3-[{3-[(2,6-dipyrrolidin-1-ylpyrimidin-4-yl)amino]propyl}(methyl)amino]propyl}urea;
N-(4-chlorophenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-[4-(piperidin-1-ylsulfonyl)phenyl]thiourea;
N-{2-[{2-[(2,6-dipyrrolidin-1-ylpyrimidin-4-yl)amino]ethyl}(methyl)amino]ethyl}-N′-(pentafluorophenyl)thiourea;
N-{2-[{2-[(2,6-dipyrrolidin-1-ylpyrimidin-4-yl)amino]ethyl}(methyl)amino]ethyl}-N′-(4-methoxyphenyl)thiourea;
N-{2-[{2-[(2,6-dipyrrolidin-1-ylpyrimidin-4-yl)amino]ethyl}(methyl)amino]ethyl}-N′-(4-phenoxyphenyl)thiourea;
N-{2-[{2-[(2,6-dipyrrolidin-1-ylpyrimidin-4-yl)amino]ethyl}(methyl)amino]ethyl}-N′-(3-fluorophenyl)thiourea;
N-(2,4-difluorophenyl)-N′-{2-[{2-[(2,6-dipyrrolidin-1-ylpyrimidin-4-yl)amino]ethyl}(methyl)amino]ethyl}thiourea;
N-(3,5-difluorophenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-(4-nitrophenyl)thiourea;
N-(4-tert-butylphenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-(3-nitrophenyl)thiourea;
N-(4,6-dimethylpyrimidin-2-yl)-4-{[({2-[{2-[(2,6-dipyrrolidin-1-ylpyrimidin-4-yl)amino]ethyl}(methyl)amino]ethyl}amino)carbonothioyl]amino}benzenesulfonamide;
N-{2-[{2-[(2,6-dipyrrolidin-1-ylpyrimidin-4-yl)amino]ethyl}(methyl)amino]ethyl}-N′-[4-(methylthio)phenyl]thiourea;
N-(4-{[3-chloro-5-(trifluoromethyl)pyridin-2-yl]thio}phenyl)-N′-{2-[{2-[(2,6-dipyrrolidin-1-ylpyrimidin-4-yl)amino]ethyl}(methyl)amino]ethyl}thiourea;
N-[4-chloro-3-(trifluoromethyl)phenyl]-N′-{2-[{2-[(2,6-dipyrrolidin-1-ylpyrimidin-4-yl)amino]ethyl}(methyl)amino]ethyl}thiourea;
N-(3,4-dichlorophenyl)-N′-{2-[{2-[(2,6-dipyrrolidin-1-ylpyrimidin-4-yl)amino]ethyl}(methyl)amino]ethyl}thiourea;
N-(4-chloro-3-fluorophenyl)-N′-{2-[{2-[(2,6-dipyrrolidin-1-ylpyrimidin-4-yl)amino]ethyl}(methyl)amino]ethyl}thiourea;
N-[4-chloro-3-(trifluoromethyl)phenyl]-N′-{5-[(2,6-dipyrrolidin-1-ylpyrimidin-4-yl)amino]pentyl}thiourea;
N-(4-chlorophenyl)-N′-{5-[(2,6-dipyrrolidin-1-ylpyrimidin-4-yl)amino]pentyl}thiourea;
4-{[({2-[{2-[(2,6-dipyrrolidin-1-ylpyrimidin-4-yl)amino]ethyl}(methyl)amino]ethyl}amino)carbonothioyl]amino}-N-methylbenzenesulfonamide;
N-{4-[(4-bromophenyl)sulfonyl]phenyl}-N′-{2-[{2-[(2,6-dipyrrolidin-1-ylpyrimidin-4-yl)amino]ethyl}(methyl)amino]ethyl}thiourea;
4-{[({2-[{2-[(2,6-dipyrrolidin-1-ylpyrimidin-4-yl)amino]ethyl}(methyl)amino]ethyl}amino)carbonothioyl]amino}benzenesulfonamide;
4-{[({2-[{2-[(2,6-dipyrrolidin-1-ylpyrimidin-4-yl)amino]ethyl}(methyl)amino]ethyl}amino)carbonothioyl]amino}-N-phenylbenzenesulfonamide;
N-(3,4-difluorophenyl)-N′-{2-[{2-[(2,6-dipyrrolidin-1-ylpyrimidin-4-yl)amino]ethyl}(methyl)amino]ethyl}thiourea;
N-(4-chlorophenyl)-N′-{2-[{2-[(2,6-dipiperidin-1-ylpyrimidin-4-yl)amino]ethyl}(methyl)amino]ethyl}thiourea;
N-{2-[(2-[2,6-bis(diethylamino)pyrimidin-4-yl]amino ethyl)(methyl)amino]ethyl}-N′-(4-chlorophenyl)thiourea;
N-(4-cyanophenyl)-N′-{2-[{2-[(2,6-dipyrrolidin-1-ylpyrimidin-4-yl)amino]ethyl}(methyl)amino]ethyl}thiourea;
   or a pharmaceutically acceptable salt thereof.

Depending on the nature of the R1, R2, W, R3, R4, and R5 groups, the compounds of the invention may be prepared according to the reaction schemes described below.

1) Preparation of the Intermediate Having the Formula (IV):

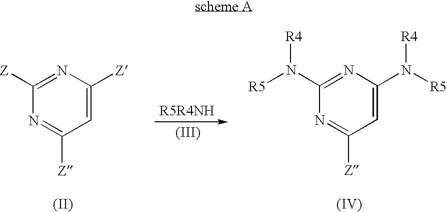

The diaminopyrimidine derivatives of general formula (IV) can be prepared according to the method described by Bundy et al. in Journal of Medicinal Chemistry, 1995, 35, 4161-4163 by reacting, for example, compound (II) wherein z, z′ and z″ represent a halogen atom and preferably a chlorine atom, with the amine compound of general formula (III) wherein R4 and R5 are as hereinbefore defined, at a temperature between −5° C. and 5° C. (preferably 0° C.), in an inert solvent such as tetrahydrofuran.

In the particular case where R4 and R5 both represent a methyl group and in the particular case where R4 and R5 represent a hydrogen atom and an ethyl group, the conditions for preparing derivatives of formula (IV) are as described by Atri et al. in Journal of Medicinal Chemistry, 1984, 27, 1621-1629. The reaction is performed at a temperature between 30° C. and 50° C. (preferably 40° C.) in an inert polar solvent such as ethanol.

2) Preparation of the Compound of General Formula (I) Wherein R1 and R2 Independently Represent a Hydrogen Atom or an Alkyl Group:

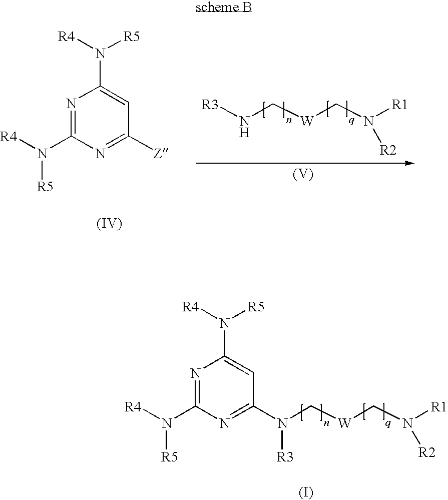

As described in scheme B hereinabove, the compounds of general formula (Ia) wherein R2, R3, R4, R5, W, n and q are as hereinbefore defined and R1 represents a hydrogen atom or an alkyl group, can be obtained, for example, by heating the compound of formula (IV) wherein z″ represents a halogen atom and preferably a chlorine atom, with a large excess of the diamine compound (V) to a temperature between 150° C. and 250° C. (preferably 190° C.) or by microwave heating.

The compounds having the general formula (Ia)

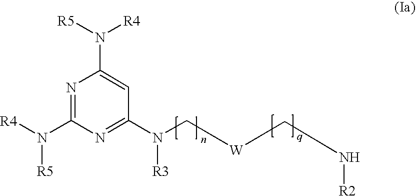

wherein R2, R3, R4, R5, W, n and q are as hereinbefore defined and R1 is a hydrogen, and obtained as described hereinabove, are used as the starting product in the reaction schemes below.

3) Preparation of the Compound of General Formula (I) Wherein R1 Represents —C(═Y)—NHR8 (Compound Ib):

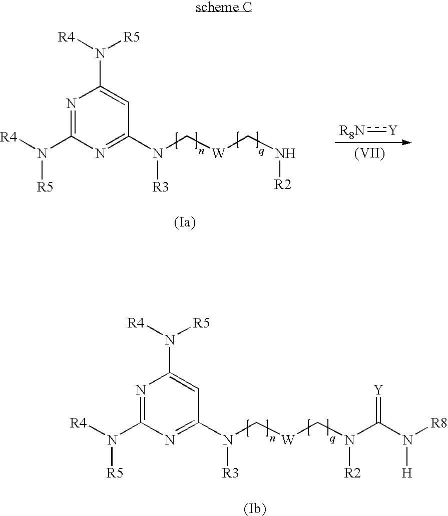

The derivatives of general formula (Ib) wherein R2, R3, R4, R5, W, n, q, and R8 are as hereinbefore defined and Y represents a sulfur or oxygen atom, can be prepared according to the method described in scheme C by reacting compound (Ia) with the isocyanate or isothiocyanate compound of general formula (VII) at a temperature between 10° C. and 30° C. (preferably 20° C.) in an inert polar solvent such as dichloromethane, 1,2-dichloromethane or dimethylformamide.

4) Preparation of the Compound of General Formula (I) Wherein R1 Represents —C(═S)—NHC(═O)R8 (Compound Ic):

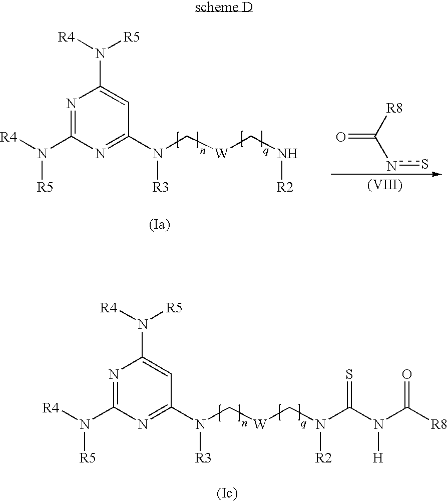

As described in scheme D hereinabove, the compounds having the general formula (Ic) where R2, R3, R4, R5, W, n, q, and R8 are as hereinbefore defined can be easily obtained under operating conditions similar to those described hereinabove for compound (Ib) and by using carboxy-isothiocyanate derivatives of formula (VIII).

5) Preparation of the Compound of General Formula (I) Wherein R1 Represents —C(═N—CN)—NHR8 (Compound Id):

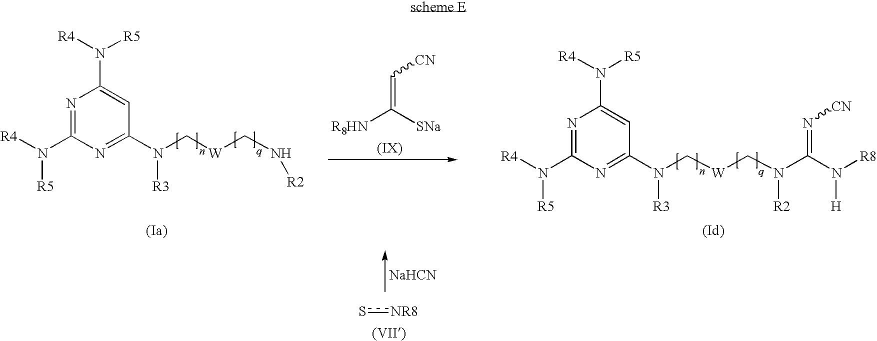

As described in scheme E above, the compounds of general formula (Id) wherein R2, R3, R4, R5, W, n, q, Y, and R8 are as hereinbefore defined, can be obtained by heating the aminopyrimidine derivative of general formula (Ia) with the cyanoethylene derivative in its salt form having the general formula (IX) at the reflux temperature of a polar solvent such as tetrahydrofuran. The salt derivative of formula (IX) can be obtained by reacting the isothiocyanate derivative of general formula (VII′) and the sodium cyanamide compound in a polar solvent such as ethanol, at a temperature between 10° C. and 30° C. (preferably 20° C.).

6) Preparation of the Compound of General Formula (I) Wherein R1 Represents —C(═O)—R9 (Compound Ie):

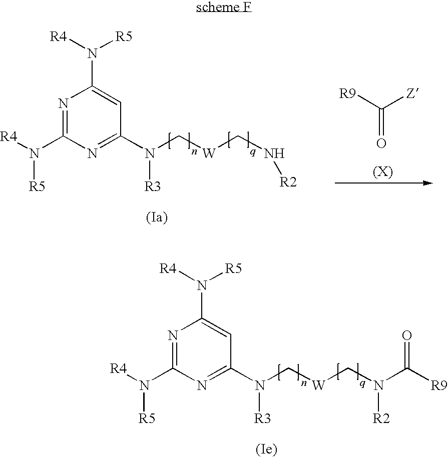

As described in scheme F above, the compounds of general formula (Ie) wherein R2, R3, R4, R5, W, n, q, Y, and R9 are as hereinbefore defined, can be obtained for example by condensation of the compound of formula (Ia) onto the acyl halide compound of general formula (X) wherein z′ represents a halogen atom and preferably a chlorine atom, in the presence of an inorganic acid scavenger such as a tertiary amine compound for example triethylamine or diisopropylethylamine at a temperature between 10° C. and 30° C. (preferably 20° C.) in an inert solvent such as dichloromethane or ethyl ether, according to methods known to the person skilled in the art.

These same compounds of general formula (Ie) can also be prepared under similar conditions to peptide coupling, by reacting carboxylic acid of general formula (XI) with compound (Ia) at a temperature between 10° C. and 30° C. (preferably 20° C.) in an inert solvent such as dichloromethane or 1,2-dichloromethane, as described in scheme G below.

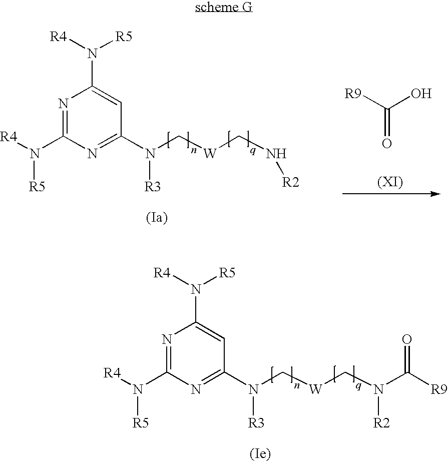

7) Preparation of the Compound of General Formula (I) Wherein R1 Represents —SO₂R10 (Compound If):

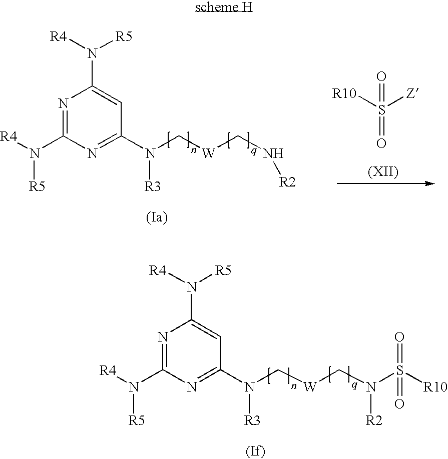

The derivatives of general formula (If) wherein R2, R3, R4, R5, W, n, q, and R10 are as hereinbefore defined, can be prepared according to the method described in scheme H hereinabove, by reacting compound (Ia) with the arylsulfonyl halide compound of formula (XII) wherein z′ represents a halogen atom and preferably a chlorine atom, in the presence of an inorganic acid scavenger such as a tertiary amine compound for example triethylamine or diisopropylethylamine, in a polar solvent such as dichloromethane, 1,2-dichloromethane or dimethylformamide at a temperature between 10° C. and 30° C. (preferably 20° C.).

The invention also concerns a process for preparing a compound of general formula (I) as hereinabove defined, comprising the following steps:
A) the compound of general formula (II)

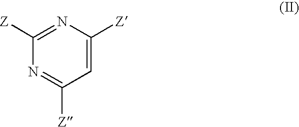

wherein z, z′ and z″ represent a halogen atom,
is reacted with an amine of general formula R5R4NH wherein R4 and R5 are as hereinbefore defined, at a temperature between −5° C. and 5° C., in an inert solvent to form the compound of general formula (IV)

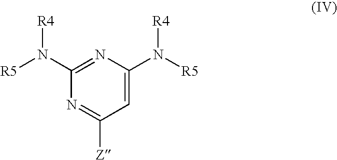

wherein R4 and R5 are as hereinbefore defined and z″ represents a halogen atom;
B) the resulting compound of general formula (IV) is then reacted with the diamine of general formula R3HN—(CH₂)ₙ—W—(CH₂)_{q}—NR1R2 wherein R3, W, n and q are as hereinbefore defined, and R1 and R2 independently represent a hydrogen atom or an alkyl group,
   by heating to a temperature between 150° C. and 250° C., to form the compound of general formula (I) wherein R1 and R2 independently represent a hydrogen atom or an alkyl group;
C) and to obtain the compounds of general formula (I) wherein R1 is neither a hydrogen atom nor an alkyl group, the corresponding compound wherein R1 is a hydrogen atom as obtained hereinabove can be reacted with:
   either an isocyanate or isothiocyanate compound of general formula R8NCY, wherein Y represents a sulfur or oxygen atom and R8 is as hereinabove defined, at a temperature between 10° C. and 30° C. in a solvent selected from dichloromethane, 1,2-dichloromethane or dimethylformamide to give the compound of general formula (I) wherein R1 represents —C(═Y)—NHR8 (compound Ib);

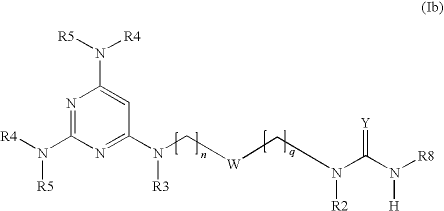

or a carboxy-isothiocyanate derivative of general formula R8C(O)NCS, wherein R8 is as hereinabove defined, at a temperature between 10° C. and 30° C. in a solvent selected from dichloromethane, 1,2-dichloromethane or dimethylformamide, to give the compound of general formula (I) wherein R1 represents —C(═S)—NHC(═O)R8 (compound Ic);

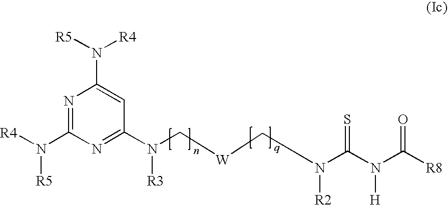

or a cyanoethylene derivative in salt form of general formula (IX), wherein R8 is as hereinabove defined,

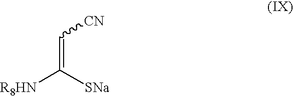

at the reflux temperature of a polar solvent, to give the compound of general formula (I) wherein R1 represents —C(═N—CN)—NHR8 (compound Id),

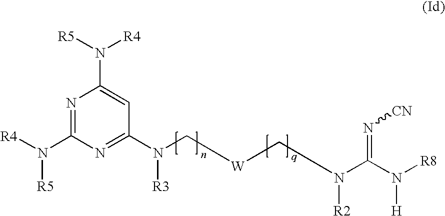

or an acyl halide compound of general formula R9C(O)_{z}′, wherein z′ represents a halogen atom and R9 is as hereinabove defined, in the presence of a tertiary amine at a temperature between 10° C. and 30° C. in an inert solvent, or alternatively a compound of general formula R9CO₂H wherein R9 is as hereinabove defined, in the presence of a peptide coupling agent, at a temperature between 10° C. and 30° C. (preferably 20° C.) in an inert solvent to give the compound of general formula (I) wherein R1 represents —C(═O)—R9 (compound Ie);

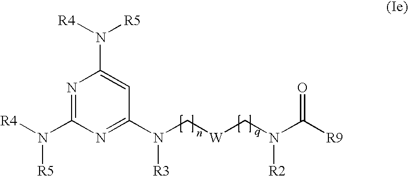

or an arylsulfonyl halide compound of formula R10SO₂z′, wherein z′ represents a halogen atom and R10 is as hereinabove defined, in the presence of a tertiary amine in a solvent selected from dichloromethane, 1,2-dichloromethane or dimethylformamide at a temperature between 10° C. and 30° C. to give the compound of general formula (I) wherein R1 represents —SO₂—R10 (compound If)

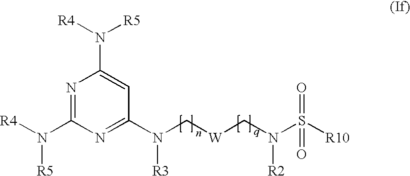

The invention also relates to a process for preparing compounds of general formula (Ia) wherein R2, R3, R4, R5, W, n and q are as hereinabove defined, and that can be obtained, for example, by microwave heating the compound of formula (IV) to a high temperature with a large excess of the diamine compound (V) wherein R1 is a hydrogen, to form the pyrimidine monoamine derivative of general formula (Ia).

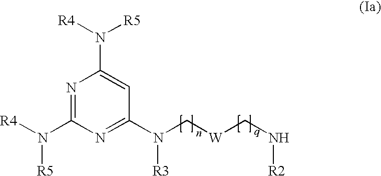

The invention also relates to an industrial compound selected from:
2,4-diazetidin-1-yl-6-chloropyrimidine;
6-chloro-N,N′-bis[2-(dimethylamino)ethyl]-N,N′-dimethylpyrimidine-2,4-diamine.

The compounds of the present invention having the general formula (I) have interesting pharmacological properties: they have Cdc25 phosphatase-inhibiting activity. They can therefore be used in various therapeutic applications.

The present invention also relates to a pharmaceutical composition containing a compound of general formula (I) as hereinabove defined, or a pharmaceutically acceptable salt of such a compound, as an active substance, with at least one pharmaceutically acceptable excipient.

The present invention also relates to a compound of general formula (I) as hereinabove defined or a pharmaceutically acceptable salt thereof as a drug.

The present invention also relates to the use of a compound of general formula (I) as hereinabove defined or a pharmaceutically acceptable salt thereof, to prepare a drug intended for the treatment or prevention of a disease or disorder selected from the following diseases or the following disorders: cancers, cancerous proliferative diseases, noncancerous proliferative diseases, neurodegenerative diseases, parasitic diseases, viral infections, spontaneous alopecia, alopecia induced by exogenous products, radiation-induced alopecia, autoimmune diseases, graft rejection, inflammatory diseases or allergies.

Preferably, the present invention concerns the use of a compound of general formula (I) as hereinabove defined or a pharmaceutically acceptable salt thereof, to prepare a drug intended for the treatment or prevention of cancer.

More preferably the present invention concerns the use of a compound of general formula (I) as hereinabove defined or a pharmaceutically acceptable salt thereof, to prepare a drug intended for the treatment or prevention of cancer, said cancer being selected from cancer of the colon, rectum, stomach, lung, pancreas, kidney, testicle, breast, uterus, ovary, prostate, skin, bone, spinal cord, neck, tongue, head as well as sarcomas, carcinomas, fibroadenomas, neuroblastomas, leukaemias and melanomas.

The compound of general formula (I) or the salt thereof used according to the invention may be in the form of a solid, for example powders, granules, tablets, capsules, liposomes or suppositories. Suitable solid bases may be, for example, calcium phosphate, magnesium stearate, talc, sugars, lactose, dextrin, starch, gelatin, cellulose, methylcellulose, sodium carboxymethylcellulose, polyvinylpyrrolidine and wax.

The compound of general formula (I) or the salt thereof used according to the invention or the combination according to the invention may also exist in liquid form, for example, solutions, emulsions, suspensions or syrups. Suitable liquid bases may be, for example, water, organic solvents such as glycerol or glycols, or blends thereof, in varying proportions, in water.

The compound of general formula (I) or its salt used according to the invention or the combination according to the invention can be administered topically, orally, parenterally, by intramuscular injection, by subcutaneous injection etc.

The anticipated dose of a product according to the present invention for the treatment of the diseases or disorders mentioned hereinabove varies depending on the method of administration, the age and the body weight of the subject to be treated as well as the subject's condition, and in the end will be decided by the treating doctor or veterinarian. Such a quantity determined by the treating doctor or veterinarian is referred to herein as the “therapeutically effective quantity”.

As an indication only, the envisaged dose of a drug according to the invention is between 0.1 mg and 10 g depending on the type of active compound used.

### EXPERIMENTAL PART

The NMR analyses of examples 1 through 90 were performed on a 400 MHz Bruker-Avance II spectrometer.

The compounds are characterised by their molecular (MH+) peak determined by mass spectrometry (MS), a single quadrupole mass spectrometer (Micromass, Platform model) equipped with an electrospray source is used with a resolution of 0.8 Da (50% valley). For examples 1 through 90 below, the elution conditions corresponding to the indicated results are as follows: elution with an acetonitrile-water-trifluoroacetic acid mixture 50-950-0.2 (A) for 1 minute, switching from mixture (A) to an acetonitrile-water mixture 950-50 (B) by a linear gradient over a period of 7.5 minutes, followed by elution with pure mixture B for 2 minutes.

Using the definitions given hereinbefore for the variable groups R1, R2, R3, R4, R5, n, q, and W, the compounds of the invention can be prepared according to the different procedures described hereinabove.

The examples are presented to illustrate the above procedures and should under no circumstances be considered to limit the scope of the invention.

### Example 1

### N-{2-[(2,6-dipyrrolidin-1-ylpyrimidin-4-yl)amino]ethyl}-N-methylethane-1,2-diamine

### 1-1) 4-chloro-2,6-dipyrrolidin-1-ylpyrimidine

At 0° C., the 2,4,6-trichloropyrimidine compound (30 g, 164 mmol) is added to a solution containing the pyrrolidine compound (44 ml, 524 mmol) in 60 ml of tetrahydrofuran. The reaction mixture is stirred for 2 hours at this temperature then for 12 hours at 23° C. Next 15 ml of pyridine is added and stirring is maintained for one half-day. 60 ml of water are added, then the reaction mixture is extracted with 3×30 ml of dichloromethane. The organic phase is poured into ice-cold water then neutralised with a saturated solution of sodium bicarbonate then with a saturated solution of sodium chloride. The organic phase is dried over sodium sulfate and the solvent is then eliminated using a rotary evaporator. The resulting oil is purified by chromatography on a Biotage type silica column (eluent: ethyl acetate-heptane: 0-100 to 5-95) and a solid is obtained in the form of a white powder. The yield of the reaction is 66%.

¹H-NMR (δ ppm, DMSO): 1.84-1.87 (m, 8H); 3-3.39 (m, 8H); 5.74 (s, 1H)

Observed MH+=253.20; theoretical M=252.12

Melting point: 84-86° C.

### 1-2) N-{2-[(2,6-dipyrrolidin-1-ylpyrimidin-4-yl)amino]ethyl}-N-methylethane-1,2-diamine

In a sealed glass tube suitable for microwave heating, the 4-chloro-2,6-dipyrrolidin-1-ylpyrimidine compound as prepared in section (1-1) (0.8 g, 3.2 mmol) and N-methyl ethylenediamine (3.3 ml, 26 mmol) are heated to 190° C. in a microwave oven (Biotage, Emrys Optimizer) for 3600 seconds. When the reaction is complete, 20 ml of water are added then the reaction mixture is extracted with ethyl acetate and washed with 3×20 ml of water. The organic phase is then dried over sodium sulfate and evaporated to dryness, then about 10 ml of heptane is added to the oil obtained. After stirring the solid obtained is filtered with a sintered-glass filter. A solid is obtained in the form of a white powder. The yield of the reaction is 69%.

¹H-NMR (δ ppm, DMSO): 1.61 (se, 2H); 1.87-1.94 (m, 8H); 2.23-2.25 (m, 3H); 2.43-2.46 (m, 2H); 2.58-2.61 (m, 2H); 2.76-2.79 (m, 2H); 3.28-3.30 (m, 2H) 3.42-3.54 (m, 8H); 4.75 (s, 1H); 4.80-4.85 (m, 1H)

Observed MH+=334.35; theoretical M=333.26

Melting point: 67-69° C.

### Example 2

### N-(3-aminopropyl)-N′-(2,6-dipyrrolidin-1-ylpyrimidin-4-yl)-N-methylpropane-1,3-diamine

This compound is synthesised according to the method presented for example 1 using the compound synthesised in section 1-1)

¹H-NMR (δ ppm, DMSO): 1.60-1.67 (m, 2H); 1.73-1.80 (m, 2H) 1.5-2 (me, 2H); 1.86-1.92 (m, 8H); 2.22 (s, 3H); 2.38-2.46 (m, 4H); 2.74-2.76 (m, 2H); 3.21-3.26 (m, 2H); 3.43-3.53 (m, 8H); 4.71 (s, 2H)

Observed MH+=362.40; theoretical M=361.53

### Example 3

### N-(2,6-dipyrrolidin-1-ylpyrimidin-4-yl)-N,N′-dimethyl-N′-[3-(methylamino)propyl]propane-1,3-diamine

This compound is synthesised according to the method presented for example 1 using the compound synthesised in section 1-1).

¹H-NMR (δ ppm, CDCl₃): 1.66-1.76 (m, 4H); 1.87-1.93 (m, 8H); 2.20 (s, 3H); 2.33-2.59 (m, 4H); 2.60 (s, 3H); 2.59-2.61 (m, 2H); 2.96 (s, 3H); 3.41-3.54 (m, 10H); 4.74 (s, 1H)

Observed MH+=390.40; theoretical M=389.58

### Example 4

### N-(2,6-dipyrrolidin-1-ylpyrimidin-4-yl)pentane-1,5-diamine

This compound is synthesised according to the method presented for example 1 using the compound synthesised in section 1-1).

¹H-NMR (δ ppm, DMSO): 1.27-1.35 (m, 4H); 1.43-1.48 (m, 3H); 1.81-1.85 (m, 8H); 2.43-2.46 (m, 2H); 3.10-3.14 (m, 2H); 3.28-3.39 (m, 9H); 4.70 (s, 1H); 5.99 (se, 1H)

Observed MH+=319.30; theoretical M=318.46

### Example 5

### N′-(2,6-dipyrrolidin-1-ylpyrimidin-4-yl)-N,N-dimethylpentane-1,5-diamine

This compound is synthesised according to the method presented for example 1 using the compound synthesised in section 1-1).

¹H-NMR (δ ppm, CDCl₃): 1.36-1.42 (m, 2H); 1.49-1.53 (m, 2H); 1.58-1.66 (m, 2H); 1.89-1.93 (m, 8H); 2.24-2.27 (m, 8H); 3.15-3.20 (m, 2H); 3.43-3.53 (m, 8H); 4.36 (se, 1H); 4.70 (s, 1H)

Observed MH+=347.40; theoretical M=346.52

### Example 6

### N′-(2,6-dipyrrolidin-1-ylpyrimidin-4-yl)-N,N-diethylpentane-1,5-diamine

This compound is synthesised according to the method presented for example 1 using the compound synthesised in section 1-1).

¹H-NMR (δ ppm, CDCl₃): 1.02 (t, 6H); 1.36-1.42 (m, 2H); 1.49-1.53 (m, 2H); 1.58-1.66 (m, 2H); 1.87-1.93 (m, 8H); 2.40-2.44 (m, 2H); 2.49-2.55 (m, 4H); 3.15-3.20 (m, 2H); 3.43-3.53 (m, 8H); 4.34 (se, 1H); 4.71 (s, 1H)

Observed MH+=375.40; theoretical M=374.57

### Example 7

### N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea

A mixture containing N-{2-[(2,6-dipyrrolidin-1-ylpyrimidin-4-yl)amino]ethyl}-N-methylethane-1,2-diamine as prepared in section 1-2) (0.1 g, 0.3 mmol) and 4-chloro-3-trifluoromethylphenylisocyanate (0.066 g, 0.3 mmol) in 3 ml of dichloromethane is stirred for 5 hours at 23° C. 3 ml of ether is added and the reaction mixture is stirred for fifteen minutes. The solid formed is filtered using a sintered-glass filter and washed with ether. After drying, a solid is obtained in the form of a white powder. The yield of the reaction is 66%.

¹H-NMR (δ ppm, DMSO): 1.76-1.81 (m, 8H); 2.24 (s, 3H); 2.43-2.50 (m, 4H); 3.15-3.37 (m, 12H); 4.71 (s, 1H); 5.85 (se, 1H); 6.23-6.26 (se, 1H); 7.50-7.54 (m, 2H), 8.03-804 (se, 1H); 9.11 (s, 1H)

Observed MH+=555.33; theoretical M=554.25

Melting point: 149-151° C.

### Example 7a

### N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea hydrochloride

The N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea (0.3 g, 0.54 mmol) as prepared in example 7 is dissolved in 10 ml of methanol. At 23° C., a 1M hydrochloric acid solution in ether (3.25 ml, 3.2 mmol) is added to this solution, then stirred for 2 hours at this temperature. The excess hydrochloric acid is removed using the evaporator then it is triturated in ether. The solid obtained is filtered using a sintered-glass filter, washing with ether. After drying, a beige powder is obtained.

¹H-NMR (δ ppm, DMSO): 1.92-2.02 (m, 8H); 2.25 (m, 2H); 3.06 (m, 3H); 3.33-3.44 (m, 16H); 4.90 (s, 1H); 7.20-7.30 (m, 2H); 7.58 (se, 1H); 7.87 (s, 1H); 8.23 (s, 1H); 8.99 (s, 1H)

Observed MH+=555.20; theoretical M=554.25

Melting point: 203-205° C.

### Example 7b

### N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea sulfate

The N-4-chloro-3-(trifluoromethyl)phenyl-N′-{2-{2-(2,6-dipyrrolidin-1-ylpyrimidin-4-yl)aminoethyl}(methyl)aminoethyl}urea (0.13 g, 0.26 mmol) as prepared in example 7 is dissolved in 1 ml of dimethylformamide. At 23° C., a 1M sulfuric acid solution (0.13 ml, 0.13 mmol) is added to this solution, then stirred for 30 minutes at this temperature. 5 ml of water is added and the solid obtained is filtered using a sintered-glass filter, washing with water. After drying, a white powder is obtained.

¹H-NMR (δ ppm, DMSO): 1.72-1.86 (m, 8H); 2.66 (s, 3H); 3.02-3.10 (m, 4H); 3.33-3.44 (m, 13H); 4.90 (s, 1H); 7.11 (se, 1H); 7.36-7.52 (m, 3H); 7.98 (s, 1H); 9.66 (s, 1H)

Observed MH+=555.18; theoretical M=554.25

Melting point: 179-199° C.

### Example 7c

### N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea tartarate [Translator's Note: Probably Tartrate]

The N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea (0.3 g, 0.6 mmol) as prepared in example 7 is dissolved in 3 ml of dimethylformamide. At 23° C. a 1M tartaric acid solution (0.6 ml, 0.6 mmol) is added to this solution, then stirred for 2 hours at this temperature. 7 ml of water is added and the solid obtained is filtered using a sintered-glass filter, washing with water. After drying, a white powder is obtained.

¹H-NMR (δ ppm, DMSO): 1.63-1.82 (m, 8H); 2.31 (s, 3H); 2.64-2.70 (m, 4H); 3.24-3.48 (m, 13H); 4.17 (s, 2H); 4.76 (s, 1H); 6.22 (se, 1H); 6.54 (se, 1H); 7.49-7.56 (m, 2H); 8.04 (s, 1H); 9.32 (s, 1H)

Observed MH+=555.16; theoretical M=554.25

Melting point: 138-145° C.

### Example 7d

### N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea citrate

The N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea (0.21 g, 0.38 mmol) as prepared in example 7 is dissolved in 2 ml of dimethylformamide. At 23° C. a 1M citric acid solution (0.38 ml, 0.38 mmol) is added to this solution, then stirred for 2 hours at this temperature. 5 ml of water is added and the solid obtained is filtered using a sintered-glass filter, washing with water. After drying, a white powder is obtained.

¹H-NMR (δ ppm, DMSO): 1.79-1.84 (m, 8H); 2.48-2.78 (m, 11H); 3.24-3.48 (m, 13H); 4.78 (s, 1H); 6.25 (se, 1H); 6.36 (se, 1H); 7.50-7.56 (m, 2H); 8.01 (s, 1H); 9.16 (s, 1H)

Observed MH+=555.17; theoretical M=554.25

Melting point: 95-126° C.

Compounds 8, 9, 10, 11, 12, 13, 14, 15, 16, 17, 18, 19, 20 and 21 were synthesised using a method analogous to the one described in example 7.

### Example 8

### N-benzyl-N′-{2-[{2-[(2,6-dipyrrolidin-1-ylpyrimidin-4-yl)amino]ethyl}(methyl)amino]ethyl}urea

¹H-NMR (δ ppm, CDCl₃): 1.79-1.92 (m, 8H); 2.24 (s, 3H); 2.46-2.62 (m, 4H); 3.18-3.40 (m, 12H); 4.32-4.33 (m, 2H); 4.70 (s, 1H); 5.07 (se, 1H); 5.45 (se, 1H); 5.72 (se, 1H), 7.25-7.30 (m, 5H)

Observed MH+=467.40; theoretical M=466.63

Melting point: 87-89° C.

### Example 9

### N-(tert-butyl)-N′-{2-[{2-[(2,6-dipyrrolidin-1-ylpyrimidin-4-yl)amino]ethyl}(methyl)amino]ethyl}urea

¹H-NMR (δ ppm, DMSO): 1.20 (s, 9H); 1.79-1.85 (m, 8H); 2.20 (s, 3H); 2.32-2.44 (m, 5H); 3.01-3.03 (m, 2H); 3.28-3.40 (m, 9H); 4.76 (s, 1H); 5.59 (se, 1H); 5.75 (se, 1H); 5.88 (se, 1H)

Observed MH+=433.40; theoretical M=432.61

Melting point: 107-109° C.

### Example 10

### N-{2-[{2-[(2,6-dipyrrolidin-1-ylpyrimidin-4-yl)amino]ethyl}(methyl)amino]ethyl}-N′-2-thienylurea

¹H-NMR (δ ppm, CDCl₃): 1.90-1.95 (m, 8H); 2.26 (s, 3H); 2.51-2.65 (m, 4H); 3.20-3.54 (m, 12H); 4.69 (s, 1H); 5.27 and 5.85 (2 se, 1H); 6.39 (se, 1H); 6.72-6.77 (m, 2H), 8.80 (se, 1H)

Observed MH+=459.30; theoretical M=458.63

Melting point: 133-135° C.

### Example 11

### N-{2-[{2-[(2,6-dipyrrolidin-1-ylpyrimidin-4-yl)amino]ethyl}(methyl)amino]ethyl}-N-[(2R)-1,2,3,4-tetrahydronaphthalen-2-yl]urea

¹H-NMR (δ ppm, CDCl₃): 1.73-1.92 (m, 10H); 2.26 (s, 3H); 2.49-2.74 (m, 7H); 3.17-3.38 (m, 12H); 4.67 (s, 1H); 4.92-5.60 (m, 4H); 7.09-7.27 (m, 5H)

Observed MH+=507.40; theoretical M=506.69

Melting point: 93-95° C.

### Example 12

### N-cyclopentyl-N′-{2-[{2-[(2,6-dipyrrolidin-1-ylpyrimidin-4-yl)amino]ethyl}(methyl)amino]ethyl}urea

¹H-NMR (δ ppm, CDCl₃): 1.26-1.63 (m, 8H); 1.86-1.93 (m, 8H); 2.26 (s, 3H); 2.46-2.62 (m, 4H); 3.19-3.98 (m, 12H); 3.98-3.99 (m, 1H); 4.72 (s, 1H); 4.93-5.20 (m, 3H)

Observed MH+=445.40; theoretical M=444.62

Melting point: 119-121° C.

### Example 13

### N-(3,5-dimethylisoxazol-4-yl)-N′-{2-[{2-[(2,6-dipyrrolidin-1-ylpyrimidin-4-yl)amino]ethyl}(methyl)amino]ethyl}urea

¹H-NMR (δ ppm, CDCl₃): 1.76-2.58 (m, 21H); 3.20-3.41 (m, 12H); 4.73 (s, 1H); 5.18 (se, 1H); 5.65 (se, 1H); 6.92 (se, 1H)

Observed MH+=472.40; theoretical M=471.61

Melting point: 95-97° C.

### Example 14

### N-{2-[{2-[(2,6-dipyrrolidin-1-ylpyrimidin-4-yl)amino]ethyl}(methyl)amino]ethyl}-N′-(2-furylmethyl)urea

¹H-NMR (δ ppm, CDCl₃): 1.80-1.94 (m, 8H); 2.24 (s, 3H); 2.38-2.62 (m, 4H); 3.19-3.51 (m, 12H); 4.31-4.33 (m, 2H); 4.70 (s, 1H); 5.18 (se, 1H); 5.45 (se, 1H); 5.80 (se, 1H); 6.20 (d, 2H); 7.27-7.29 (m, 1H)

Observed MH+=457.40; theoretical M=456.59

Melting point: 103-105° C.

### Example 15

### N-2,1,3-benzothiadiazol-4-yl-N′-{2-[{2-[(2,6-dipyrrolidin-1-ylpyrimidin-4-yl)amino]ethyl}(methyl)amino]ethyl}urea

¹H-NMR (δ ppm, DMSO): 1.88-1.93 (m, 8H); 2.28 (s, 3H); 2.60-2.69 (m, 4H); 3.18-3.57 (m, 12H); 4.66 (s, 1H); 5.56 (se, 1H); 6.72 (se, 1H); 7.50-7.57 (m, 2H); 8.30-8.32 (d, 1H); 9.00 (s, 1H)

Observed MH+=511.40; theoretical M=510.67

Melting point: 126-128° C.

### Example 16

### N-(4-chlorophenyl)-N′-{2-[{2-[(2,6-dipyrrolidin-1-ylpyrimidin-4-yl)amino]ethyl}(methyl)amino]ethyl}urea

¹H-NMR (δ ppm, CDCl₃): 1.88-1.95 (m, 8H); 2.29 (s, 3H); 2.50-2.66 (m, 4H); 3.20-3.52 (m, 12H); 4.72 (s, 1H); 5.17 (se, 1H); 5.72 (se, 1H); 7.14-7.31 (m, 4H); 7.87 (se, 1H)

Observed MH+=487.36; theoretical M=486.26

Melting point: 71-73° C.

### Example 17

### N-(3,4-dichlorophenyl)-N′-{2-[{2-[(2,6-dipyrrolidin-1-ylpyrimidin-4-yl)amino]ethyl}(methyl)amino]ethyl}urea

¹H-NMR (δ ppm, DMSO): 1.76-1.82 (m, 8H); 2.23 (s, 3H); 2.42-2.50 (m, 4H); 3.13-3.37 (m, 12H); 4.71 (s, 1H); 5.75 (se, 1H); 6.22 (se, 1H); 7.20-7.43 (m, 2H); 7.81 (s, 1H); 8.94 (s, 1H)

Observed MH+=521.29; theoretical M=520.22

Melting point: 138-139° C.

### Example 18

### N-{2-[{2-[(2,6-dipyrrolidin-1-ylpyrimidin-4-yl)amino]ethyl}(methyl)amino]ethyl}-N-ethylurea

¹H-NMR (δ ppm, DMSO): 0.96 (t, 3H); 1.79-1.83 (m, 8H); 2.19 (s, 3H); 2.34-2.49 (m, 2H); 2.94-3.07 (m, 4H); 3.23-3.39 (m, 12H); 4.75 (s, 1H); 5.66 (se, 1H); 5.87 (se, 2H)

Observed MH+=405.30; theoretical M=404.56

Melting point: 127-129° C.

### Example 19

### N-(4-chlorophenyl)-N′-{5-[(2,6-dipyrrolidin-1-ylpyrimidin-4-yl)amino]pentyl}urea

¹H-NMR (δ ppm, DMSO): 1.03-1.32 (m, 6H); 1.78-1.84 (m, 8H); 3.05-3.38 (m, 12H); 4.70 (s, 1H); 6.00 (se, 1H); 6.13 (se, 1H); 7.30 (dd, 4H); 8.51 (s, 1H)

Observed MH+=472.35; theoretical M=471.25

Melting point: 192-193° C.

### Example 20

### N-[4-chloro-3-(trifluoromethyl)phenyl]-N′-(2-{2-[(2,6-dipyrrolidin-1-ylpyrimidin-4-yl)amino]ethoxy}ethyl)urea

¹H-NMR (δ ppm, CDCl₃): 1.6 (m, 5H); 2 (m, 8H); 3.30-3.71 (m, 12H); 4.65 (s, 1H); 7.05 (se, 1H); 7.30 (m, 1H); 7.72 (m, 1H); 7.89 (d, 1H); 9.03 (se, 1H)

Observed MH+=542.20; theoretical M=541.22

Melting point: 79-81° C.

### Example 21

### N-[4-chloro-3-(trifluoromethyl)phenyl]-N′-{3-[{3-[(2,6-dipyrrolidin-1-ylpyrimidin-4-yl)amino]propyl}(methyl)amino]propyl}urea

Observed MH+=583.21; theoretical M=582.28

### Example 21a

### N-[4-chloro-3-(trifluoromethyl)phenyl]-N′-{3-[{3-[(2,6-dipyrrolidin-1-ylpyrimidin-4-yl)amino]propyl}(methyl)amino]propyl}urea hydrochloride

This compound is synthesised according to the method presented for example 7a using the compound synthesised in example 21

¹H-NMR (δ ppm, DMSO): 1.83-1.96 (m, 12H); 2.73 (s, 3H); 3.0-3.53 (m, 14H); 5.05 (s, 1H); 6.76 (se, 1H); 7.51-8.06 (m, 4H); 9.53 (s, 1H); 10.36 (se, 1H); 11.56 (se, 1H)

Observed MH+=583.17; theoretical M=582.28

Melting point: 115-117° C.

### Example 22

### N-(4-chlorophenyl)-N′-{2-[{2-[(2,6-dipyrrolidin-1-ylpyrimidin-4-yl)amino]ethyl}(methyl)amino]ethyl}thiourea

A mixture containing N-{2-[(2,6-dipyrrolidin-1-ylpyrimidin-4-yl)amino]ethyl}-N-methylethane-1,2-diamine as prepared in section (1-2) (0.1 g, 0.3 mmol) and 4-chlorophenylisothiocyanate (0.051 g, 0.3 mmol) in 3 ml of dichloromethane is stirred for two hours at 23° C. The solid obtained is filtered using a sintered-glass filter. After washing with ether then drying in a vacuum chamber, a white powder is obtained. The yield of the reaction is 53%.

¹H-NMR (δ ppm, DMSO): 1.77-1.98 (m, 8H); 2.30 (s, 3H); 2.61-2.67 (m, 4H); 3.22-3.71 (m, 12H); 4.69 (s, 1H); 5.20 (se, 1H); 7.05 (se, 1H); 7.27-7.35 (m, 4H), 8.3 (se, 1H)

Observed MH+=503.29; theoretical M=502.24

Melting point: 127-129° C.

### Example 22a

### N-(4-chlorophenyl)-N′-{2-[{2-[(2,6-dipyrrolidin-1-ylpyrimidin-4-yl)amino]ethyl}(methyl)amino]ethyl}thiourea hydrochloride

This compound is synthesised according to the method presented for example 7a using the compound synthesised in example 22

¹H-NMR (δ ppm, DMSO): 1.86-1.93 (m, 8H); 2.88 (s, 3H); 3.9-3.36 (m, 16H); 5.24 (se, 1H); 7.35-7.7.49 (dd, 4H); 7.73 (se, 1H); 8.2 (se, 1H); 10.15 (se, 1H); 10.70 (se, 1H); 11.42 (se, 1H)

Observed MH+=503.19; theoretical M=502.24

Melting point: 211-213° C.

The compounds 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 67, 76 and 77 were synthesised according to a method analogous to the one described in example 22.

### Example 23

### N-{2-[{2-[(2,6-dipyrrolidin-1-ylpyrimidin-4-yl)amino]ethyl}(methyl)amino]ethyl}-N′-[4-(trifluoromethoxy)phenyl]thiourea

¹H-NMR (δ ppm, CDCl₃): 1.76-1.95 (m, 8H); 2.31 (s, 3H); 2.61-2.68 (m, 4H); 3.21-3.71 (m, 12H); 4.70 (s, 1H); 5.05 (se, 1H); 7.16 (d, 3H); 7.42 (d, 2H); 8.60 (se, 1H)

Observed MH+=553.40; theoretical M=552.68

Melting point: 118-120° C.

### Example 24

### N-{2-[{2-[(2,6-dipyrrolidin-1-ylpyrimidin-4-yl)amino]ethyl}(methyl)amino]ethyl}-N′-(4-fluorophenyl)thiourea

¹H-NMR (δ ppm, CDCl₃): 1.79-1.96 (m, 8H); 2.29 (s, 3H); 2.60-2.66 (m, 4H); 3.21-3.71 (m, 12H); 4.69 (s, 1H); 4.95 (se, 1H); 7.02-7.32 (m, 4H); 8.30 (d, 2H)

Observed MH+=487.30 theoretical M=486.66

Melting point: 115-117° C.

### Example 25

### N-{2-[{2-[(2,6-dipyrrolidin-1-ylpyrimidin-4-yl)amino]ethyl}(methyl)amino]ethyl}-N′-[4-(trifluoromethyl)phenyl]thiourea

¹H-NMR (δ ppm, CDCl₃): 1.77-1.93 (m, 8H); 2.32 (s, 3H); 2.63-2.70 (m, 4H); 3.23-3.73 (m, 12H); 4.70 (s, 1H); 4.25 (se, 1H); 7.35 (se, 1H); 7.53 (d, 2H); 7.61 (d, 2H); 9.00 (se, 1H)

Observed MH+=537.40; theoretical M=536.67

Melting point: 87-89° C.

### Example 26

### N-{2-[{2-[(2,6-dipyrrolidin-1-ylpyrimidin-4-yl)amino]ethyl}(methyl)amino]ethyl}-N′-pyridin-3-ylthiourea

¹H-NMR (δ ppm, CDCl₃): 1.76-1.97 (m, 8H); 2.32 (s, 3H); 2.63-2.70 (m, 4H); 3.23-3.73 (m, 12H); 4.70 (s, 1H); 4.25 (se, 1H); 7.35 (se, 1H); 7.53 (d, 2H); 7.61 (d, 2H); 9.00 (se, 1H)

Observed MH+=470.30; theoretical M=469.66

Melting point: 107-109° C.

### Example 27

### N-{2-[{2-[(2,6-dipyrrolidin-1-ylpyrimidin-4-yl)amino]ethyl}(methyl)amino]ethyl}-N′-[4-(piperidin-1-ylsulfonyl)phenyl]thiourea

¹H-NMR (δ ppm, CDCl₃): 1.40-1.43 (m, 2H); 1.61-1.67 (m, 4H); 1.81-1.94 (m, 8H); 2.31 (s, 3H); 2.64-2.70 (m, 4H); 2.97-3.00 (m, 4H); 3.22-3.74 (m, 12H); 4.70 (s, 1H); 5.30 (se, 1H); 7.35 (se, 1H); 7.64 (d, 2H); 7.78 (d, 2H); 9.30 (se, 1H)

Observed MH+=616.40; theoretical M=615.87

Melting point: 120-122° C.

### Example 28

### N-{2-[{2-[(2,6-dipyrrolidin-1-ylpyrimidin-4-yl)amino]ethyl}(methyl)amino]ethyl}-N′-ethylthiourea

¹H-NMR (δ ppm, CDCl₃): 1.15 (t, 3H); 1.76-1.97 (m, 8H); 2.32 (s, 3H); 2.63-2.70 (m, 4H); 3.20 (q, 2H); 3.45-3.73 (m, 12H); 4.70 (s, 1H); 4.95 (se, 1H); 6.75 (se, 1H); 6.90 (se, 1H)

Observed MH+=421.30; theoretical M=420.63

Melting point: 82-84° C.

### Example 29

### N-{2-[{2-[(2,6-dipyrrolidin-1-ylpyrimidin-4-yl)amino]ethyl}(methyl)amino]ethyl}-N′-(2-furylmethyl)thiourea

¹H-NMR (δ ppm, CDCl₃): 1.76-1.97 (m, 8H); 2.32 (s, 3H); 2.63-2.70 (m, 4H); 3.23-3.73 (m, 12H); 4.70 (s, 1H); 4.75 (m, 2H); 5.15 (se, 1H); 6.30 (d, 2H); 6.80 (se, 1H); 7.30 (d, 1H)

Observed MH+=421.30; theoretical M=324.38

Melting point: 99-101° C.

### Example 30

### N-{2-[{2-[(2,6-dipyrrolidin-1-ylpyrimidin-4-yl)amino]ethyl}(methyl)amino]ethyl}-N′-(6-phenoxypyridin-3-yl)thiourea

¹H-NMR (δ ppm, CDCl₃): 1.76-1.93 (m, 8H); 2.33 (s, 3H); 2.61-2.70 (m, 4H); 3.23-3.70 (m, 12H); 4.70 (s, 1H); 5.10 (se, 1H); 6.86 (d, 1H); 7.12-7.41 (m, 6H); 7.91-7.99 (m, 2H); 8.50 (se, 1H)

Observed MH+=562.30; theoretical M=561.76

Melting point: 104-106° C.

### Example 31

### N-{2-[{2-[(2,6-dipyrrolidin-1-ylpyrimidin-4-yl)amino]ethyl}(methyl)amino]ethyl}-N′-(tetrahydrofuran-2-ylmethyl)thiourea

¹H-NMR (δ ppm, CDCl₃): 1.62 (qd, 1H); 1.88-1.94 (m, 13H), 2.26 (s, 3H); 2.57-2.64 (m, 4H); 3.26-4.09 (m, 17H), 4.70 (s, 1H); 5.21 (se, 1H); 7.00 (se, 1H)

Observed MH+=477.20; theoretical M=476.69

Melting point: 120-122° C.

### Example 32

### N-{2-[{2-[(2,6-dipyrrolidin-1-ylpyrimidin-4-yl)amino]ethyl}(methyl)amino]ethyl}-N′-(6-morpholin-4-ylpyridin-3-yl)thiourea

¹H-NMR (δ ppm, CDCl₃): 1.77-1.95 (m, 8H); 2.29 (s, 3H); 2.59-2.65 (m, 4H); 3.23-3.81 (m, 20H); 4.86 (s, 1H); 4.95 (se, 1H); 6.63 (d, 1H); 6.88 (se, 1H); 7.58 (d, 1H); 8.02 (s, 1H); 8.10 (se, 1H)

Observed MH+=555.40; theoretical M=554.76

Melting point: 116-118° C.

### Example 33

### N-{2-[{2-[(2,6-dipyrrolidin-1-ylpyrimidin-4-yl)amino]ethyl}(methyl)amino]ethyl}-N′-[4-(1,3-oxazol-5-yl)phenyl]thiourea

¹H-NMR (δ ppm, CDCl₃): 1.79-1.93 (m, 8H); 2.30 (s, 3H); 2.63-2.66 (m, 4H); 3.23-3.72 (m, 12H); 4.68 (s, 1H); 5.10 (se, 1H); 7.28 (m, 1H); 7.50-7.60 (m, 4H); 7.89 (s, 1H); 8.20 (se, 1H)

Observed MH+=536.40; theoretical M=535.72

Melting point: 104-106° C.

### Example 34

### N-{2-[{2-[(2,6-dipyrrolidin-1-ylpyrimidin-4-yl)amino]ethyl}(methyl)amino]ethyl}-N′-(pentafluorophenyl)thiourea

¹H-NMR (δ ppm, CDCl₃): 1.80-1.94 (m, 8H); 2.34 (s, 3H); 2.59-2.65 (m, 4H); 3.2-3.75 (m, 12H); 4.76 (s, 1H); 5.10 (se, 1H); 7.50 (m, 1H)

Observed MH+=559.30; theoretical M=558.62

Melting point: 92-94° C.

### Example 35

### N-{2-[{2-[(2,6-dipyrrolidin-1-ylpyrimidin-4-yl)amino]ethyl}(methyl)amino]ethyl}-N′-(4-methoxyphenyl)thiourea

¹H-NMR (δ ppm, CDCl₃): 1.76-1.95 (m, 8H); 2.25 (s, 3H); 2.57-2.61 (m, 4H); 3.19-3.77 (m, 15H); 4.56 (se, 1H); 4.66 (s, 1H); 6.70 (se, 1H); 6.91 (d, 2H); 7.20 (d, 2H); 7.80 (se, 1H)

Observed MH+=499.40; theoretical M=498.70

Melting point: 127-129° C.

### Example 36

### N-{2-[{2-[(2,6-dipyrrolidin-1-ylpyrimidin-4-yl)amino]ethyl}(methyl)amino]ethyl}-N′-(4-phenoxyphenyl)thiourea

¹H-NMR (δ ppm, CDCl₃): 1.78-1.93 (m, 8H); 2.29 (s, 3H); 2.60-2.65 (m, 4H); 3.21-3.70 (m, 12H); 4.68 (s, 1H); 4.80 (se, 1H); 6.95-7.35 (m, 10H); 8.20 (se, 1H)

Observed MH+=561.40; theoretical M=560.77

Melting point: 79-81° C.

### Example 37

### N-{2-[{2-[(2,6-dipyrrolidin-1-ylpyrimidin-4-yl)amino]ethyl}(methyl)amino]ethyl}-N′-1-naphthylthiourea

¹H-NMR (δ ppm, CDCl₃): 1.63-1.93 (m, 8H); 2.12 (s, 3H); 2.36-2.52 (m, 4H); 3.01-3.68 (m, 12H); 4.40 (se, 1H); 4.60 (s, 1H); 6.80 (se, 1H); 7.52 (m, 4H); 7.81-7.91 (m, 4H)

Observed MH+=519.40; theoretical M=518.73

Melting point: 87-89° C.

### Example 38

### N-{2-[{2-[(2,6-dipyrrolidin-1-ylpyrimidin-4-yl)amino]ethyl}(methyl)amino]ethyl}-N′-(3,4,5-trimethoxyphenyl)thiourea

¹H-NMR (δ ppm, CDCl₃): 1.79-1.93 (m, 8H); 2.30 (s, 3H); 2.61-2.66 (m, 4H); 3.21-3.70 (m, 12H); 3.82 (s, 9H); 4.68 (s, 1H); 4.80 (se, 1H); 6.61 (se, 2H); 7.00 (se, 1H); 8.3 (se, 1H)

Observed MH+=559.40; theoretical M=558.75

Melting point: 109-111° C.

### Example 39

### N-{2-[{2-[(2,6-dipyrrolidin-1-ylpyrimidin-4-yl)amino]ethyl}(methyl)amino]ethyl}-N′-(3-fluorophenyl)thiourea

Observed MH+=487.30; theoretical M=486.66

Melting point: 118-120° C.

### Example 40

### N-(2,4-difluorophenyl)-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.80-1.95 (m, 8H); 2.30 (s, 3H); 2.64-2.68 (m, 4H); 3.22-3.74 (m, 12H); 4.68 (s, 1H); 5.30 (se, 1H); 6.77 (m, 1H); 6.97 (m, 1H); 7.7 (m, 1H); 7.95 (m, 1H); 8.80 (se, 1H)

Observed MH+=505.30; theoretical M=504.65

Melting point: 124-126° C.

### Example 41

### N-(3,5-difluorophenyl)-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.84-1.94 (m, 8H); 2.30 (s, 3H); 2.63-2.69 (m, 4H); 3.21-3.71 (m, 12H); 4.69 (s, 1H); 5.30 (se, 1H); 6.51 (m, 1H); 7.24 (m, 2H); 7.45 (se, 1H); 9.20 (se, 1H)

Observed MH+=505.30; theoretical M=504.65

Melting point: 124-126° C.

### Example 42

### N-{2-[{2-[(2,6-dipyrrolidin-1-ylpyrimidin-4-yl)amino]ethyl}(methyl)amino]ethyl}-N′-(2-fluorophenyl)thiourea

¹H-NMR (δ ppm, CDCl₃): 1.80-1.94 (m, 8H); 2.29 (s, 3H); 2.62-2.66 (m, 4H); 3.22-3.72 (m, 12H); 4.67 (s, 1H); 5.10 (se, 1H); 7.12 (m, 3H); 7.25 (se, 1H); 7.80 (se, 1H); 8.40 (se, 1H)

Observed MH+=487.40; theoretical M=486.27

Melting point: 105-107° C.

### Example 43

### N-{2-[{2-[(2,6-dipyrrolidin-1-ylpyrimidin-4-yl)amino]ethyl}(methyl)amino]ethyl}-N′-(4-nitrophenyl)thiourea

¹H-NMR (δ ppm, DMSO): 1.84-1.94 (m, 8H); 2.31 (s, 3H); 2.65-2.70 (m, 4H); 3.22-3.74 (m, 12H); 4.70 (s, 1H); 5.35 (se, 1H); 7.50 (se, 1H); 7.82 (m, 2H); 8.12 (d, 2H); 9.50 (se, 1H)

Observed MH+=514.35; theoretical M=513.26

Melting point: 146-148° C.

### Example 44

### N-(4-tert-butylphenyl)-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.30 (s, 9H); 1.79-1.94 (m, 8H); 2.27 (s, 3H); 2.58-2.62 (m, 4H); 3.19-3.69 (m, 12H); 4.63 (s, 1H); 4.70 (se, 1H); 6.91 (se, 1H); 7.24 (m, 2H); 7.39 (m, 2H); 8.10 (se, 1H)

Observed MH+=525.42; theoretical M=524.31

Melting point: 192-194° C.

### Example 45

### N-{2-[{2-[(2,6-dipyrrolidin-1-ylpyrimidin-4-yl)amino]ethyl}(methyl)amino]ethyl}-N′-[4-(4-nitrophenoxy)phenyl]thiourea

¹H-NMR (δ ppm, CDCl₃): 1.76-1.94 (m, 8H); 2.30 (s, 3H); 2.63-2.68 (m, 4H); 3.22-3.73 (m, 12H); 4.69 (s, 1H); 4.90 (se, 1H); 7.00-7.08 (m, 5H); 7.45-7.47 (m, 2H); 8.19 (d, 2H); 8.70 (se, 1H)

Observed MH+=606.35; theoretical M=605.29

Melting point: foam

### Example 46

### N-{2-[{2-[(2,6-dipyrrolidin-1-ylpyrimidin-4-yl)amino]ethyl}(methyl)amino]ethyl}-N′-(4-fluorobenzyl)thiourea

¹H-NMR (δ ppm, CDCl₃): 1.78-1.94 (m, 8H); 2.22 (s, 3H); 2.56-2.63 (m, 4H); 3.17-3.67 (m, 12H); 4.68 (m, 3H); 5.00 (se, 1H); 6.80 (se, 1H); 7.00 (m, 2H); 7.24 (m, 2H)

Observed MH+=501.39; theoretical M=500.28

Melting point: 64-66° C.

### Example 47

### N-[2-(2,4-difluorophenoxy)pyridin-3-yl]-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.27-2.04 (m, 8H); 2.27 (s, 3H); 2.66-2.70 (m, 4H); 3.15-3.84 (m, 12H); 6.78-6.86 (m, 2H); 6.97-7.00 (m, 1H); 7.19-7.27 (m, 1H); 7.79 (se, 1H); 8.17 (se, 1H); 8.57 (d, 1H); 9.01 (se, 1H); 9.82 (se, 1H); 10.8 (se, 1H)

Observed MH+=598.39; theoretical M=597.28

Melting point: 108-110° C.

### Example 48

### N-{2-[{2-[(2,6-dipyrrolidin-1-ylpyrimidin-4-yl)amino]ethyl}(methyl)amino]ethyl}-N′-[4-(1H-pyrazol-1-yl)phenyl]thiourea

¹H-NMR (δ ppm, CDCl₃): 1.77-1.96 (m, 8H); 2.30 (s, 3H); 2.62-2.67 (m, 4H); 3.22-3.73 (m, 12H); 4.69 (s, 1H); 5.00 (se, 1H); 6.45 (s, 1H); 7.10 (se, 1H); 7.48 (d, 2H); 7.65-7.71 (m, 3H); 7.88 (s, 1H); 8.50 (se, 1H)

Observed MH+=535.40; theoretical M=534.73

Melting point: 116-118° C.

### Example 49

### N-{2-[{2-[(2,6-dipyrrolidin-1-ylpyrimidin-4-yl)amino]ethyl}(methyl)amino]ethyl}-N′-(3-nitrophenyl)thiourea

¹H-NMR (δ ppm, CDCl₃): 1.78-1.94 (m, 8H); 2.32 (s, 3H); 2.64-2.71 (m, 4H); 3.23-3.73 (m, 12H); 4.70 (s, 1H); 5.30 (se, 1H); 7.42 (t, 2H); 7.96 (d, 1H); 8.09 (d, 1H); 8.25 (s, 1H); 9.50 (se, 1H)

Observed MH+=514.31; theoretical M=513.26

Melting point: 125-127° C.

### Example 50

### N-(4,6-dimethylpyrimidin-2-yl)-4-{[({2-[{2-[(2,6-dipyrrolidin-1-ylpyrimidin-4-yl)amino]ethyl}(methyl)amino]ethyl}amino)carbonothioyl]amino}benzenesulfonamide

¹H-NMR (δ ppm, CDCl₃): 1.75-1.96 (m, 8H); 2.17 (s, 3H); 2.26 (s, 6H); 2.61-2.63 (m, 5H); 3.18-3.64 (m, 12H); 4.60 (se, 1H); 6.40 (se, 1H); 6.50 (s, 1H); 7.66 (m, 2H); 7.92 (m, 2H); 8.00 (se, 1H); 9.50 (se, 1H)

Observed MH+=654.38; theoretical M=653.30

Melting point: 129° C. (foam)

### Example 51

### N-{2-[{2-[(2,6-dipyrrolidin-1-ylpyrimidin-4-yl)amino]ethyl}(methyl)amino]ethyl}-N′-[4-(methylthio)phenyl]thiourea

¹H-NMR (δ ppm, CDCl₃): 1.80-1.94 (m, 8H); 2.28 (s, 3H); 2.45 (s, 3H); 2.60-2.65 (m, 4H); 3.21-3.70 (m, 12H); 4.67 (s, 1H); 4.90 (se, 1H); 6.90 (se, 1H); 7.23-7.27 (m, 4H); 8.30 (se, 1H)

Observed MH+=515.38; theoretical M=514.27

Melting point: 67-69° C.

### Example 52

### N-(4-{[3-chloro-5-(trifluoromethyl)pyridin-2-yl]thio}phenyl)-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.79-1.93 (m, 8H); 2.33 (s, 3H); 2.63-2.69 (m, 4H); 3.25-3.72 (m, 12H); 4.68 (s, 1H); 4.80 (se, 1H); 7.10 (se, 1H); 7.49-7.53 (m, 4H); 7.76 (s, 1H); 8.39 (s, 2H)

Observed MH+=680.36; theoretical M=679.22

Melting point: 97-99° C.

### Example 53

### N-(6-chloropyridin-3-yl)-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.90-1.94 (m, 8H); 2.29 (s, 3H); 2.64-2.71 (m, 4H); 3.22-3.74 (m, 12H); 4.66 (s, 1H); 7.21 (d, 1H); 8.36 (se, 2H); 10.20 (se, 1H); 10.80 (se, 1H)

Observed MH+=504.30; theoretical M=503.23

Melting point: 96-98° C.

### Example 54

### N-[4-chloro-3-(trifluoromethyl)phenyl]-N′-{2-[{2-[(2,6-dipyrrolidin-1-ylpyrimidin-4-yl)amino]ethyl}(methyl)amino]ethyl}thiourea

¹H-NMR (δ ppm, DMSO): 1.76-1.94 (m, 8H); 2.33 (s, 3H); 2.62-2.70 (m, 4H); 3.23-3.70 (m, 12H); 4.70 (s, 1H); 5.30 (se, 1H); 7.30 (se, 1H); 7.39 (d, 1H); 7.64 (se, 1H); 7.78-7.79 (m, 1H); 9.00 (se, 1H)

Observed MH+=571.25; theoretical M=570.23

Melting point: 117-119° C.

### Example 55

### N-(3,4-dichlorophenyl)-N′-{2-[{2-[(2,6-dipyrrolidin-1-ylpyrimidin-4-yl)amino]ethyl}(methyl)amino]ethyl}thiourea

¹H-NMR (δ ppm, DMSO): 1.78-1.96 (m, 8H); 2.32 (s, 3H); 2.61-2.69 (m, 4H); 3.23-3.70 (m, 12H); 4.70 (s, 1H); 5.20 (se, 1H); 7.21-7.50 (m, 4H); 8.60 (se, 1H)

Observed MH+=535.25; theoretical M=536.20

Melting point: 148-150° C.

### Example 56

### N-(4-chloro-3-fluorophenyl)-N′-{2-[{2-[(2,6-dipyrrolidin-1-ylpyrimidin-4-yl)amino]ethyl}(methyl)amino]ethyl}thiourea

¹H-NMR (δ ppm, DMSO): 1.76-1.96 (m, 8H); 2.33 (s, 3H); 2.60-2.69 (m, 4H); 3.23-3.70 (m, 12H); 4.70 (s, 1H); 5.00 (se, 1H); 7.05-7.20 (m, 2H); 7.32-7.35 (m, 2H); 8.40 (se, 1H)

Observed MH+=521.28; theoretical M=520.23

Melting point: 146-148° C.

### Example 57

### N-[4-chloro-3-(trifluoromethyl)phenyl]-N′-{5-[(2,6-dipyrrolidin-1-ylpyrimidin-4-yl)amino]pentyl}thiourea

¹H-NMR (δ ppm, CDCl₃): 1.47-1.94 (m, 14H), 3.29-3.63 (m, 12H); 4.30 (se, 1H); 4.72 (s, 1H); 6.30 (se, 1H); 7.48 (d, 1H); 7.64-7.65 (m, 2H); 7.80 (se, 1H)

Observed MH+=556.20; theoretical M=555.22

Melting point: 85-87° C.

### Example 58

### N-(4-chlorophenyl)-N′-{5-[(2,6-dipyrrolidin-1-ylpyrimidin-4-yl)amino]pentyl}thiourea

¹H-NMR (δ ppm, CDCl₃): 1.42-1.95 (m, 14H), 3.19-3.66 (m, 12H); 4.50 (se, 1H); 4.70 (s, 1H); 6.10 (se, 1H); 7.20 (d, 2H); 7.36 (d, 2H); 7.70 (se, 1H)

Observed MH+=488.26; theoretical M=487.23

Melting point: 80-82° C.

### Example 59

### 4-{[({2-[{2-[(2,6-dipyrrolidin-1-ylpyrimidin-4-yl)amino]ethyl}(methyl)amino]ethyl}amino)carbonothioyl]amino}-N-methylbenzenesulfonamide

¹H-NMR (δ ppm, CDCl₃): 1.82-1.95 (m, 8H); 2.31 (s, 3H); 2.64-2.69 (m, 4H); 2.65 (s, 3H); 3.22-3.73 (m, 12H); 4.20 (se, 1H); 4.70 (s, 1H); 5.30 (se, 1H); 7.50 (se, 1H); 7.74-7.76 (m, 4H); 9.50 (se, 1H)

Observed MH+=562.26; theoretical M=561.27

Melting point: 102-104° C.

### Example 60

### N-{4-[(4-bromophenyl)sulfonyl]phenyl}-N′-{2-[{2-[(2,6-dipyrrolidin-1-ylpyrimidin-4-yl)amino]ethyl}(methyl)amino]ethyl}thiourea

¹H-NMR (δ ppm, DMSO): 1.76-1.94 (m, 8H); 2.30 (s, 3H); 2.62-2.69 (m, 4H); 3.22-3.70 (m, 12H); 4.70 (s, 1H); 5.30 (se, 1H); 7.61-7.81 (m, 7H); 9.00 (se, 1H)

Observed MH+=689.10; theoretical M=686.18

Melting point: 118-120° C.

### Example 61

### 4-{[({2-[{2-[(2,6-dipyrrolidin-1-ylpyrimidin-4-yl)amino]ethyl}(methyl)amino]ethyl}amino)carbonothioyl]amino}benzenesulfonamide

¹H-NMR (δ ppm, CDCl₃): 1.80-1.94 (m, 8H); 2.30 (s, 3H); 2.65-2.66 (m, 4H); 3.23-3.71 (m, 12H); 4.00 (se, 2H); 4.68 (s, 1H); 5.30 (se, 1H); 7.57 (m, 2H); 7.75 (m, 2H)

Observed MH+=548.28; theoretical M=547.25

Melting point: 140° C. (foam)

### Example 62

### 4-{[({2-[{2-[(2,6-dipyrrolidin-1-ylpyrimidin-4-yl)amino]ethyl}(methyl)amino]ethyl}amino)carbonothioyl]amino}-N-phenylbenzenesulfonamide

¹H-NMR (δ ppm, CDCl₃): 1.80-1.95 (m, 8H); 2.27 (s, 3H); 2.62-2.67 (m, 4H); 3.20-3.71 (m, 12H); 4.67 (s, 1H); 5.30 (se, 1H); 7.03-7.27 (m, 10H); 7.70 (se, 1H); 7.62 (se, 1H)

Observed MH+=624.20; theoretical M=623.28

Melting point: 146-148° C.

### Example 63

### N˜6˜-{2-[(2-aminoethyl)(methyl)amino]ethyl}-N˜2˜,N˜4˜-bis[2-(dimethylamino)ethyl]-N˜2˜,N˜4˜-dimethylpyrimidine-2,4,6-triamine

### 63-1) 6-chloro-N,N′-bis[2-(dimethylamino)ethyl]-N,N′-dimethylpyrimidine-2,4-diamine

This intermediate is synthesised according to the method presented in section 1-1)

¹H-NMR (δ ppm, CDCl₃): 2.28 (s, 12H); 2.45-2.51 (m, 4H); 3.01 (s, 3H); 3.13 (s, 3H); 3.31-3.71 (m, 4H); 5.7 (s, 1H)

Observed MH+=315.17; theoretical M=314.20

### 63-2) N˜6˜-{2-[(2-aminoethyl)(methyl)amino]ethyl}-N˜2˜,N˜4˜-bis[2-(dimethylamino)ethyl]-N˜2˜,N˜4˜-dimethylpyrimidine-2,4,6-triamine

This compound is synthesised according to the method presented in section 1-2)

¹H-NMR (δ ppm, CDCl₃): 1.38 (se, 2H); 2.25-2.44 (m, 15H), 2.45-2.51 (m, 4H); 3.01 (s, 3H); 3.13 (s, 3H); 3.31-3.71 (m, 4H); 5.7 (s, 1H)

Observed MH+=396.31; theoretical M=395.35

### Example 64

### N-{2-[[2-({2,6-bis[[2-(dimethylamino)ethyl](methyl)amino]pyrimidin-4-yl}amino)ethyl](methyl)amino]ethyl}-N′-(4-chlorophenyl)thiourea

This compound is synthesised according to the method presented for example 22 using the compound synthesised in section 63-2)

¹H-NMR (δ ppm, CDCl₃): 2.21-2.30 (m, 15H); 2.41-2.48 (m, 4H); 2.62-2.66 (m, 4H); 2.93-3.22 (m, 8H); 3.57-3.73 (m, 6H); 4.79 (s, 1H); 5.00 (se, 1H); 6.95 (se, 1H); 7.28-7.35 (m, 4H); 8.80 (se, 1H)

Observed MH+=565.27; theoretical M=564.32

Melting point: gum

### Example 65

### N-{2-[(2,6-dimorpholin-4-ylpyrimidin-4-yl)amino]ethyl}-N-methylethane-1,2-diamine

### 65-1) 4,4′-(6-chloropyrimidine-2,4-diyl)dimorpholine

This intermediate is synthesised according to the method presented in section 1-1)

¹H-NMR (δ ppm, CDCl₃): 3.55 (m, 4H); 3.74-3.77 (m, 12H); 5.88 (s, 1H)

Observed MH+=285.10; theoretical M=284.10

### 65-2) N-{2-[(2,6-dimorpholin-4-ylpyrimidin-4-yl)amino]ethyl}-N-methylethane-1,2-diamine

This compound is synthesised according to the method presented in section 1-2)

¹H-NMR (δ ppm, CDCl₃): 1.53 (se, 2H); 2.05 (s, 3H); 2.46 (t, 2H); 2.59 (t, 2H); 2.79 (t, 2H); 3.33 (m, 2H); 3.48 (m, 4H); 3.69-4.12 (m, 12H); 4.92-4.96 (s and se, 2H)

Observed MH+=366.29; theoretical M=365.25

### Example 66

### N-(4-chlorophenyl)-N′-{2-[{2-[(2,6-dimorpholin-4-ylpyrimidin-4-yl)amino]ethyl}(methyl)amino]ethyl}thiourea

This compound is synthesised according to the method presented for example 22 using the compound synthesised in section 65-2)

¹H-NMR (δ ppm, CDCl₃): 2.30 (s, 3H); 2.61-2.64 (s, 4H); 3.29-3.76 (m, 20H); 4.60 (se, 1H); 4.86 (s, 1H); 6.70 (se, 1H); 7.22-7.34 (m, 4H); 7.80 (se, 1H)

Observed MH+=535.20; theoretical M=534.23

Melting point: 81-83° C.

### Example 67

### N-(3,4-difluorophenyl)-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.77-1.85 (m, 8H); 2.32 (s, 3H); 2.60-2.69 (m, 4H); 3.23-3.70 (m, 12H); 4.70 (s, 1H); 5.30 (se, 1H); 7.07-7.11 (m, 3H); 7.40 (se, 1H); 8.60 (se, 1H)

Observed MH+=505.26; theoretical M=504.26

Melting point: 133-135° C.

### Example 68

### N-{2-[(2,6-dipiperidin-1-ylpyrimidin-4-yl)amino]ethyl}-N-methylethane-1,2-diamine

### 68-1) 4-chloro-2,6-dipiperidin-1-ylpyrimidine

This intermediate is synthesised according to the method presented in section 1-1)

¹H-NMR (δ ppm, CDCl₃): 1.56-1.66 (m, 12H); 3.53 (t, 4H); 3.72 (t, 4H); 5.82 (s, 1H)

Observed MH+=281.18; theoretical M=280.14

### 68-2) N-{2-[(2,6-dipiperidin-1-ylpyrimidin-4-yl)amino]ethyl}-N-methylethane-1,2-diamine

This compound is synthesised according to the method presented in section 1-2)

¹H-NMR (δ ppm, CDCl₃): 1.58-1.67 (m, 12H); 2.25 (s, 3H); 2.45 (t, 2H); 2.59 (t, 2H); 2.79 (t, 2H); 3.30 (m, 2H); 3.48-3.70 (m, 8H); 4.80 (se, 1H); 4.93 (s; 1H)

Observed MH+=362.35; theoretical M=361.29

### Example 69

### N-(4-chlorophenyl)-N′-{2-[{2-[(2,6-dipiperidin-1-ylpyrimidin-4-yl)amino]ethyl}(methyl)amino]ethyl}thiourea

This compound is synthesised according to the method presented for example 22 using the compound synthesised in section 68-2)

¹H-NMR (δ ppm, CDCl₃): 1.48-1.64 (m, 12H); 2.30 (s, 3H); 2.61-2.63 (m, 4H); 3.26-3.68 (m, 12H); 4.60 (se, 1H); 4.88 (s, 1H); 7.00 (s, 1H); 7.27-7.32 (m, 4H); 8.00 (se, 1H)

Observed MH+=531.24; theoretical M=530.27

Melting point: 76-78° C.

### Example 70

### N˜6˜-{2-[(2-aminoethyl)(methyl)amino]ethyl}-N˜2˜,N˜2˜,N˜4˜,N˜4˜-tetraethylpyrimidine-2,4,6-triamine

### 70-1) 6-chloro-N,N,N′,N′-tetraethylpyrimidine-2,4-diamine

This intermediate is synthesised according to the method presented in section 1-1)

Observed MH+=257.18; theoretical M=256.14

### 70-2) N˜6˜-{2-[(2-aminoethyl)(methyl)amino]ethyl}-N˜2˜,N˜2˜,N˜4˜,N˜4˜-tetraethylpyrimidine-2,4,6-triamine

This compound is synthesised according to the method presented in section 1-2)

¹H-NMR (δ ppm, CDCl₃): 1.14 (t, 12H); 2.26 (s, 3H); 2.45 (t, 2H); 2.59 (t, 2H); 2.75 (t, 2H); 3.30-3.39 (m, 2H); 3.45 (q, 4H); 3.54 (q, 4H); 4.68 (se, 1H); 4.81 (s, 1H)

Observed MH+=337.29; theoretical M=338.28

### Example 71

### N-{2-[(2-{[2,6-bis(diethylamino)pyrimidin-4-yl]amino}ethyl)(methyl)amino]ethyl}-N′-(4-chlorophenyl)thiourea

This compound is synthesised according to the method presented for example 22 using the compound synthesised in section 70-2)

¹H-NMR (δ ppm, CDCl₃): 1.08-1.27 (m, 12H); 2.31 (s, 3H); 2.61-2.65 (m, 4H); 3.26-3.68 (m, 12H); 4.76 (s, 1H); 4.80 (se, 1H); 7.00 (se, 1H); 7.27-7.30 (m, 4H); 8.10 (se, 1H)

Observed MH+=507.22; theoretical M=506.27

Melting point: gum

### Example 72

### N˜6˜-{2-[(2-aminoethyl)(methyl)amino]ethyl}-N˜2˜,N˜2˜,N˜4˜,N˜4˜-tetramethylpyrimidine-2,4,6-triamine

### 72-1) 6-chloro-N,N,N′,N′-tetramethylpyrimidine-2,4-diamine

This intermediate is synthesised according to the method presented in section 1-1)

¹H-NMR (δ ppm, CDCl₃): 3.04 (s, 6H); 3.13 (s, 6H); 5.76 (s, 1H)

Observed MH+=201.29; theoretical M=200.08

### 72-2) N˜6˜-{2-[(2-aminoethyl)(methyl)amino]ethyl}-N˜2˜,N˜2˜,N˜4˜,N˜4˜-tetramethylpyrimidine-2,4,6-triamine

This compound is synthesised according to the method presented in section 1-2)

¹H-NMR (δ ppm, CDCl₃): 1.81 (m, 6H); 2.24 (s, 3H); 2.45 (t, 2H); 2.59 (t, 2H); 2.76 (t, 2H); 3.00 (s, 3H); 3.09 (s, 3H); 3.30 (se, 2H); 4.83 (s and se, 2H)

Observed MH+=282.37; theoretical M=281.23

### Example 73

### N-{2-[(2-{[2,6-bis(dimethylamino)pyrimidin-4-yl]amino}ethyl)(methyl)amino]ethyl}-N′-(4-chlorophenyl)thiourea

This compound is synthesised according to the method presented for example 22 using the compound synthesised in section 72-2)

¹H-NMR (δ ppm, CDCl₃): 2.31 (s, 3H); 2.61-2.68 (m, 4H); 2.90-3.68 (m, 16H); 4.60 (se, 1H); 4.79 (s, 1H); 6.70 (se, 1H); 7.27-7.32 (m, 4H); 8.00 (se, 1H)

Observed MH+=451.27; theoretical M=450.21

Melting point: 127-129° C.

### Example 74

### N-{2-[(2,6-diazetidin-1-ylpyrimidin-4-yl)amino]ethyl}-N-methylethane-1,2-diamine

### 74-1) 2,4-diazetidin-1-yl-6-chloropyrimidine

This intermediate is synthesised according to the method presented in section 1-1)

¹H-NMR (δ ppm, CDCl₃): 2.29 (q, 2H); 2.37 (q, 2H); 4.03 (t, 4H); 4.09 (t, 4H); 5.53 (s, 1H); 3

Observed MH+=225.09; theoretical M=224.08

### 74-2) N-{2-[(2,6-diazetidin-1-ylpyrimidin-4-yl)amino]ethyl}-N-methylethane-1,2-diamine

This compound is synthesised according to the method presented in section 1-2)

Observed MH+=306.43; theoretical M=305.23

### Example 75

### N-(4-chlorophenyl)-N′-{2-[{2-[(2,6-diazetidin-1-ylpyrimidin-4-yl)amino]ethyl}(methyl)amino]ethyl}thiourea

This compound is synthesised according to the method presented for example 22 using the compound synthesised in section 74-2)

¹H-NMR (δ ppm, CDCl₃): 2.24-2.42 (m, 7H); 2.61-2.66 (m, 4H); 3.16-3.19 (m, 2H); 3.76 (m, 2H); 4.02-4.06 (m, 8H); 4.49 (s, 1H); 7.26-7.30 (m, 4H); 7.80 (se, 1H); 7.90 (se, 1H); 9.5-10 (se, 1H)

Observed MH+=475.32; theoretical M=474.21

Melting point: 146-148° C.

### Example 76

### N-[4-(dimethylamino)phenyl]-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.80-1.95 (m, 8H); 2.30 (s, 3H); 2.62-2.67 (m, 4H); 2.90 (s, 6H); 3.20-3.71 (m, 12H); 4.50 (se, 1H); 4.65 (s, 1H); 6.60 (se, 1H); 6.70 (m, 2H); 7.15 (m, 2H); 7.70 (se, 1H)

Observed MH+=512.40; theoretical M=511.74

Melting point: 127-129° C.

### Example 77

### N-(4-cyanophenyl)-N′-{2-[{2-[(2,6-dipyrrolidin-1-ylpyrimidin-4-yl)amino]ethyl}(methyl)amino]ethyl}thiourea

¹H-NMR (δ ppm, CDCl₃): 1.83-1.95 (m, 8H); 2.30 (s, 3H); 2.63-2.69 (m, 4H); 3.21-3.72 (m, 12H); 4.69 (s, 1H); 5.30 (se, 1H); 7.48-7.75 (m, 5H); 9.00-10.00 (se, 1H)

Observed MH+=494.32; theoretical M=493.27

Melting point: 107-109° C.

### Example 78

### N˜4˜-{2-[(2-aminoethyl)(methyl)amino]ethyl}-N˜2˜,N˜6˜-diethylpyrimidine-2,4,6-triamine

### 78-1) 6-chloro-N,N′-diethylpyrimidine-2,4-diamine

This intermediate is synthesised according to the method presented in section 1-1)

¹H-NMR (δ ppm, CDCl₃): 1.13 (t, 6H); 3.14-3.34 (m, 4H); 4.62-4.78 (m, 2H); 5.62 (s, 1H)

Observed MH+=200.08; theoretical M=201.23

### 78-2) N˜4˜-{2-[(2-aminoethyl)(methyl)amino]ethyl}-N˜2˜,N˜6˜-diethylpyrimidine-2,4,6-triamine

This compound is synthesised according to the method presented in section 1-2)

¹H-NMR (δ ppm, CDCl₃): 1.13 (t, 6H); 1.30-1.70 (se, 2H); 2.16 (s, 3H); 2.37 (t, 2H); 2.51 (t, 2H); 2.71 (t, 2H); 3.09-3.31 (m, 6H); 4.30-4.37 (m, 2H); 4.70 (s, 1H); 4.87 (se, 1H)

Observed MH+=282.31; theoretical M=281.23

### Example 79

### N-{2-[(2-{[2,6-bis(ethylamino)pyrimidin-4-yl]amino}ethyl)(methyl)amino]ethyl}-N′-(4-chlorophenyl)thiourea

This compound is synthesised according to the method presented for example 22 using the compound synthesised in section 78-2)

¹H-NMR (δ ppm, CDCl₃): 1.04 (t, 3H); 1.14 (t, 3H); 2.20 (s, 3H); 2.53-2.56 (m, 4H); 3.12-3.22 (m, 6H); 3.62 (m, 2H); 4.27-4.36 (m, 2H); 4.66 (s, 1H); 4.90 (se, 1H); 6.80 (se, 1H); 7.26 (s, 4H); 8.60 (se, 1H)

Observed MH+=451.25; theoretical M=450.21

Melting point: 142-144° C.

### Example 80

### N-[({2-[{2-[(2,6-dipyrrolidin-1-ylpyrimidin-4-yl)amino]ethyl}(methyl)amino]ethyl}amino)carbonothioyl]-4-methoxybenzamide

A mixture containing N-{2-[(2,6-dipyrrolidin-1-ylpyrimidin-4-yl)amino]ethyl}-N-methylethane-1,2-diamine as prepared in section (1-2) (0.1 g, 0.3 mmol) and 4-methoxybenzoyl isothiocyanate (0.058 g, 0.3 mmol) in 3 ml of dichloromethane is stirred for two hours at 23° C. The solid obtained is filtered using a sintered-glass filter. After washing with ether then drying in a vacuum chamber, a white powder is obtained. The yield of the reaction is 44%.

¹H-NMR (δ ppm, DMSO): 1.77-1.98 (m, 8H); 2.32 (s, 3H); 2.61-2.67 (m, 4H); 3.22-3.81 (m, 12H); 3.85 (s, 3H); 4.80 (s, 1H); 5.20 (se, 1H); 6.97 (d, 2H); 7.85 (d, 2H). 8.9 (se, 1H); 11.1 (se, 1H)

Observed MH+=527.30; theoretical M=526.71

Melting point: 171-173° C.

### Example 81

### N-(4-chlorophenyl)-N″-cyano-N′-{2-[{2-[(2,6-dipyrrolidin-1-ylpyrimidin-4-yl)amino]ethyl}(methyl)amino]ethyl}guanidine

### 81-1) sodium N-(4-chlorophenyl)-N′-cyanoimidothiocarbamate

A mixture containing 4-chlorophenylisothiocyanate (0.036 g, 0.2 mmol) and sodium cyanamide (0.016 g, 0.25 mmol) in 2 ml of ethanol is heated for 15 minutes at 100° C. The reaction mixture is stirred for 1 hour at 23° C. then concentrated using a rotary evaporator. The product is used in the following step without purification. The yield of the reaction is 100%.

### 81-2) N-(4-chlorophenyl)-N″-cyano-N′-{2-[{2-[(2,6-dipyrrolidin-1-ylpyrimidin-4-yl)amino]ethyl}(methyl)amino]ethyl}guanidine

A mixture containing N-{2-[(2,6-dipyrrolidin-1-ylpyrimidin-4-yl)amino]ethyl}-N-methylethane-1,2-diamine as prepared in section (1-2) (0.1 g, 0.3 mmol) and sodium N-(4-chlorophenyl)-N′-cyanoimidothiocarbamate as prepared in section (77-1) (0.050 g, 0.21 mmol) in 4 ml of tetrahydrofuran is stirred for 10 minutes at 23° C. Mercuric chloride is added over a period of 20 minutes, then stirred for a further 30 minutes. About 0.5 ml of water is added and the reaction medium is then filtered using celite. The filtrate is dried over sodium sulfate then concentrated using a rotary evaporator. The resulting oil is purified by chromatography on a Biotage type silica column (eluent: dichloromethane-MeOH: 100-0 to 95-5) and a solid is obtained in the form of a yellow powder. The yield of the reaction is 66%.

Observed MH+=511.30; theoretical M=510.273

Melting point: 120-122° C.

### Example 82

### N-{2-[{2-[(2,6-dipyrrolidin-1-ylpyrimidin-4-yl)amino]ethyl}(methyl)amino]ethyl}quinoxaline-2-carboxamide

A mixture containing N-{2-(2,6-dipyrrolidin-1-ylpyrimidin-4-yl)aminoethyl}-N-methylethane-1,2-diamine as prepared in section (1-2) (0.1 g, 0.3 mmol) and quinoxaline-2 carbonyl chloride (0.064 g, 0.33 mmol) in 2 ml of dichloromethane in the presence of diisopropyl ethylamine (0.063 ml, 0.36 mmol) is stirred for two hours at 23° C. 10 ml of water are added, then the reaction mixture is extracted with 3×10 ml of dichloromethane.

The organic phase is poured into ice-cold water then neutralised with a saturated solution of sodium bicarbonate then with a saturated solution of sodium chloride. The organic phase is dried over sodium sulfate and the solvent is then eliminated using a rotary evaporator. The resulting oil is purified by chromatography on a Biotage type silica column (eluent: dichloromethane-MeOH: 100-0 to 90-10) and a solid is obtained in the form of a pale yellow powder. The yield of the reaction is 38%.

¹H-NMR (δ ppm, DMSO): 1.72-1.80 (m, 8H); 2.30 (s, 3H); 2.49-2.675 (m, 4H); 3.15-3.50 (m, 12H); 4.64 (s, 1H); 5.77 (se, 1H); 7.92-8.00- (m, 2H); 8.14-8.19 (m, 2H), 8.86-8.88 (m, 1H); 9.45 (s, 1H)

Observed MH+=490.30; theoretical M=489.62

Melting point: 146-148° C.

The compounds 83, 84, 85 and 86 presented below were synthesised according to a method analogous to the one described in example 82.

### Example 83

### 4-benzoyl-N-{2-[{2-[(2,6-dipyrrolidin-1-ylpyrimidin-4-yl)amino]ethyl}(methyl)amino]ethyl}benzamide

¹H-NMR (δ ppm, CDCl₃): 1.79-1.93 (m, 8H); 2.35 (s, 3H); 2.67-2.68 (m, 4H); 3.30-3.57 (m, 12H); 4.68 (s, 1H); 4.80 (se, 1H); 7.20 (se, 1H); 7.47-7.87 (m, 9H)

Observed MH+=542.20; theoretical M=541.70

Melting point: 119-121° C.

### Example 84

### N-{2-[{2-[(2,6-dipyrrolidin-1-ylpyrimidin-4-yl)amino]ethyl}(methyl)amino]ethyl}-2-phenoxypropanamide

¹H-NMR (δ ppm, CDCl₃): 1.58-1.61 (m, 3H); 1.86-1.94 (m, 8H); 2.11 (s, 3H); 2.35-2.60 (m, 4H); 3.20-3.54 (m, 12H); 4.30 (se, 1H); 4.69 (s, 2H); 6.94-7.00 (m, 4H); 7.30 (m; 2H)

Observed MH+=482.40; theoretical M=481.64

Melting point: 110-112° C.

### Example 85

### N-{2-[{2-[(2,6-dipyrrolidin-1-ylpyrimidin-4-yl)amino]ethyl}(methyl)amino]ethyl}-9-oxo-9H-fluorene-4-carboxamide

¹H-NMR (δ ppm, DMSO): 1.76-1.82 (m, 8H); 2.30 (s, 3H); 2.50-2.60 (m, 4H); 3.18-3.45 (m, 12H); 4.61 (s, 1H); 5.79 (se, 1H); 7.33-7.82 (m, 7H); 8.60 (m; 1H)

Observed MH+=540.40; theoretical M=539.68

Melting point: 139-140° C.

### Example 86

### N-{2-[{2-[(2,6-dipyrrolidin-1-ylpyrimidin-4-yl)amino]ethyl}(methyl)amino]ethyl}-1-[4-(trifluoromethyl)pyrimidin-2-yl]piperidine-4-carboxamide

¹H-NMR (δ ppm, CDCl₃): 1.61-1.77 (m, 4H); 1.88-1.93 (m, 8H); 2.16 (q, 1H); 2.30 (s, 3H); 2.50-2.63 (m, 4H); 2.83 (t, 2H); 3.26-3.51 (m, 12H); 4.72-4.75 (m, 4H); 6.41 (se, 1H); 6.71 (d, 1H); 8.46 (d; 1H)

Observed MH+=591.40; theoretical M=590.69

Melting point: 171-173° C.

### Example 87

### N-{2-[{2-[(2,6-dipyrrolidin-1-ylpyrimidin-4-yl)amino]ethyl}(methyl)amino]ethyl}-4-nitrobenzenesulfonamide

A mixture containing N-{2-(2,6-dipyrrolidin-1-ylpyrimidin-4-yl)aminoethyl}-N-methylethane-1,2-diamine as prepared in section (1-2) (0.1 g, 0.3 mmol) and 2-nitro benzene sulfonyl chloride (0.073 g, 0.33 mmol) in 5 ml of dichloromethane in the presence of diisopropyl ethylamine (0.078 ml, 0.36 mmol) is stirred for two hours at 23° C. 10 ml of water are added, then the reaction mixture is extracted with 3×10 ml of dichloromethane.

The organic phase is dried over sodium sulfate and the solvent is then eliminated using a rotary evaporator. The resulting brown oil is purified by chromatography on a Biotage type silica column (eluent: dichloromethane-MeOH: 100-0 to 90-10) and a solid is obtained in the form of a pale yellow powder. The yield of the reaction is 23%.

¹H-NMR (δ ppm, DMSO): 1.79-1.986 (m, 8H); 2.08 (s, 3H); 2.37-2.38 (m, 4H); 2.90-2.91 (m, 2H); 3.15-3.39 (s, 10H); 4.73 (s, 1H); 5.88 (se, 1H); 7.88 (se, 1H); 8.025 (d, 2H), 8.34 (d, 2H)

Observed MH+=519.35; theoretical M=518.24

Melting point: 144-145° C.

The compounds 88 and 89 mentioned hereinafter were synthesised according to a method analogous to the one described in example 87.

### Example 88

### N-{2-[{2-[(2,6-dipyrrolidin-1-ylpyrimidin-4-yl)amino]ethyl}(methyl)amino]ethyl}-3-(trifluoromethyl)benzenesulfonamide

¹H-NMR (δ ppm, CDCl₃): 1.86-1.94 (m, 8H); 3.10 (s, 3H); 3.24-3.40 (m, 16H); 4.80 (se, 1H); 7.00-7.50 (se, 1H); 7.79-8.10 (m, 4H); 11-11.5 (se; 1H)

Observed MH+=542.29; theoretical M=541.24

Melting point: 203-205° C.

### Example 89

### N-{2-[{2-[(2,6-dipyrrolidin-1-ylpyrimidin-4-yl)amino]ethyl}(methyl)amino]ethyl}-4-(trifluoromethyl)benzenesulfonamide

¹H-NMR (δ ppm, CDCl₃): 1.86-1.94 (m, 8H); 3.10 (s, 3H); 3.24-3.40 (m, 16H); 4.80 (se, 1H); 7.00-7.50 (se, 1H); 7.89-7.97 (m, 4H); 11-11.5 (se; 1H)

Observed MH+=542.29; theoretical M=541.24; Melting point: 140-142° C.

### Pharmacological Study of the Compounds of the Invention:

### Test Protocols

### i) Determination of the Phosphatase Activity of the Purified Recombinant Cdc25C Enzyme:

The phosphatase activity of the MBP-Cdc25C protein is evaluated through its dephosphorylation of 3-O-methylfluorescein-phosphate (OMFP) to form 3-O-methylfluorescein (OMF), determining the fluorescence of the reaction product at 475 nm. This assay can be used to identify inhibitors of the recombinant Cdc25 enzyme. The preparation of the MBP-Cdc25C fusion protein is described in patent application PCT WO 01/44467.

The reaction is performed in 384-well plates in a final volume of 50 μl. The MBP-Cdc25C protein (prepared as described hereinabove) is stored in the following elution buffer: 20 mM Tris-HCl pH 7.4; 250 mM NaCl; 1 mM EDTA; 1 mM dithiothreitol (DTT); 10 mM maltose. It is diluted to a concentration of 60 μM in the following reaction buffer: 50 mM Tris-HCl pH 8.2; 50 mM NaCl; 1 mM DTT; 20% glycerol. The background noise is determined using with the buffer without addition of the enzyme. The products are tested at decreasing concentrations from 40 μM. The reaction is initiated by the addition of a solution OMFP to a final concentration of 500 μM (prepared immediately before use from a 12.5 mM stock solution in 100% DMSO (Sigma #M2629)). After 4 hours at 30° C. in a disposable 384-well plate, the fluorescence measured at OD 475 nm is read on a Victor² plate reader (EGG-Wallac). The concentration that produces 50% inhibition of the enzyme reaction is calculated from three independent experiments. Only the values that fall within the linear part of the sigmoid curve are used for the linear regression analysis.

### ii) Characterisation of Anti-Proliferative Activity:

By way of an example, the effect of treating two human cell lines Mia-Paca2 and DU145 with the compounds of the examples described hereinbefore will be studied. The cell lines DU145 (human prostate cancer cells) and Mia-PaCa2 (human pancreas cancer cells) were obtained from the American Tissue Culture Collection (Rockville, Md., USA). A 96-well plate was inoculated on day 0 with cells placed in 80 μl of Dulbecco's modified Eagle's medium (Gibco-Brl, Cergy-Pontoise, France) with 10% heat-inactivated foetal calf serum (Gibco-Brl, Cergy-Pontoise, France), 50000 units/1 of penicillin and 50 mg/l of streptomycin (Gibco-Brl, Cergy-Pontoise, France), and 2 mM of glutamine (Gibco-Brl, Cergy-Pontoise, France). The cells were treated on day 1 with increasing concentrations of each test compound up to 10 μM for 96 hours. At the end of this period, cell proliferation is quantified by means of a colorimetric test based on cleavage of the tetrazolium salt WST1 by the mitochondrial dehydrogenases in the viable cells, resulting in formation of formazan (Boehringer Mannheim, Meylan, France). These tests are performed in duplicate with 8 determinations per concentration tested. For each compound to be tested, the values within the linear part of the sigmoid curve were subjected to linear regression analysis and used to estimate the IC₅₀ inhibitory concentration. The products are solubilised in dimethylsulfoxide (DMSO) at a concentration of 10⁻² M and used in culture with a final DMSO concentration of 0.1%.

### Results of the Tests:

a) Results on the CDC25 Enzyme

The IC₅₀ of the compounds of examples 1 to 56, 58 to 62, 64, 66, 67, 69, 71, 73, 75 to 77, 80, 81, and 83 to 89 on the activity of the purified recombinant Cdc25-C enzyme is less than or equal to 10000 nM.

Among these compounds, the IC₅₀ of examples 1 to 24, 26 to 56, 58 to 62, 64, 67, 69, 71, 73, 77, 80, 81, and 83 to 89 is less than or equal to 5000 nM.

Among the latter, the IC₅₀ of examples 7, 10, 13 to 17, 19, 22, 26, 28, 38 to 40, 43, 44, 46, 47, 49 to 53, 59 to 62, 67, 69, 71, 77, 81, and 87 is less than or equal to 1000 nM.

b) Results on Proliferation of the Mia-Paca2 Cell Line

The IC₅₀ of the compounds of examples 7, 10, 11, 15 to 17, 19 to 27, 29, 30, 33 to 49, 51 to 58, 60, 62, 64, 67, 69, 71, 73, 75 to 77, 79, and 84 to 89 is less than or equal to 5000 nM on proliferation of the Mia-Paca2 cell line.

Among these compounds, the IC₅₀ of the examples 7, 10, 11, 20, 22 to 25, 30, 33 to 37, 39 to 41, 43 to 45, 49, 51, 52, 54 to 58, 67, 69, 71, and 77 is less than or equal to 1000 nM.

c) Results on Proliferation of the DU-145 Cell Line

The IC₅₀ of the compounds of examples 7, 10, 11, 15 to 17, 19 to 25, 27; 29, 30, 33 to 49, 51 to 58, 60, 62, 67, 69, 71, 73, 75 to 77, 88, and 89 is less than or equal to 5000 nM on the proliferation of the DU-145 cell line.

Among these compounds, the IC₅₀ of the examples 7, 20, 22, 34, 39 to 41, 43, 49, 52, 54 to 58, 67, 71 and 77 is less than or equal to 1000 nM.

1. Compound having the general formula (I) 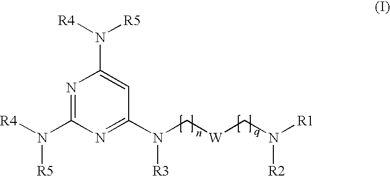
in a racemic form, an enantiomeric form or any combination thereof, wherein:
R1 represents a hydrogen atom, an alkyl group, a —C(═O)—NHR8, —C(═S)—NHR8, —C(═S)—NH—C(═O)—R8, —C(═N—CN)—NHR8, —C(═O)—R9 or —SO₂—R10 group;
R2 represents a hydrogen atom, a C₁-C₃ linear or branched alkyl group;
W represents —NR6-, —CR6R7-, an oxygen atom or a sulfur atom;
R6 and R7 represent independently a hydrogen atom or an alkyl group;
n and q are integers from 2 to 6 inclusive;
R3 represents a hydrogen atom or an alkyl group;
R4 and R5 represent independently a hydrogen atom, an alkyl group, an aminoalkyl, alkylaminoalkyl or dialkylaminoalkyl group; or alternatively R4 and R5 together with the nitrogen atom to which they are attached form a heterocycloalkyl;
R8 represents a hydrogen atom or one of the following groups:
alkyl,
cycloalkyl,
heterocycloalkylalkyl,
heteroaryl optionally substituted by one or more identical or different groups, wherein said groups are: alkyl, heterocycloalkyl, halo or aryloxy optionally substituted by one or more identical or different halogens,
heteroarylalkyl,
aryl optionally substituted by one or more identical or different groups, wherein said groups are: alkyl; alkoxy; alkylthio; dialkylamino; halo; haloalkyl; haloalkyloxy; cyano; nitro; heteroaryl; heteroarylthio optionally substituted by one or more identical or different groups, wherein said groups are: halo or haloalkyl; aryloxy optionally substituted by one or more nitro groups; arylsulfonyl optionally substituted by one or more identical or different halogens; or a —SO₂NR15R16 group;
arylalkyl optionally substituted by one or more identical or different halogens, or;
a group having the formula: 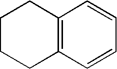
R9 represents one of the following groups:
aryl optionally substituted by one or more arylcarbonyl groups,
aryloxyalkyl optionally substituted by a C₁-C₃ alkyl group,
heteroaryl;
heterocycloalkyl optionally substituted by a heteroaryl, itself optionally substituted by a haloalkyl group;
R10 represents an aryl group optionally substituted by one or more identical or different groups, wherein said groups are: haloalkyl or nitro;
R15 and R16 may together form a heterocycloalkyl group including the nitrogen atom, or R15 and R16 independently represent a heteroaryl group optionally substituted by one or more identical or different C₁-C₃ alkyls, a C₁-C₃ alkyl group, an aryl group or a hydrogen atom;
or a pharmaceutically acceptable salt thereof.

2. The compound of claim 1, wherein R4 and R5 represent independently a hydrogen atom, an alkyl, aminoalkyl, alkylaminoalkyl or dialkylaminoalkyl group; or a pharmaceutically acceptable salt of said compound.

3. The compound of claim 1, wherein R4 and R5 together with the nitrogen atom to which they are attached form a heterocycloalkyl; or a pharmaceutically acceptable salt of said compound.

4. The compound of claim 1, wherein W represents —CR6R7-; or a pharmaceutically acceptable salt of said compound.

5. The compound of claim 4, wherein R1 represents —C(═S)—NHR8, —C(═S)—NH—C(═O)—R8 or —C(═N—CN)—NHR8; or a pharmaceutically acceptable salt of said compound.

6. The compound of claim 5, wherein:
R1 represents a —C(═S)—NHR8 group;
R2 represents a hydrogen atom;
W represents —CR6R7-;
R6 and R7 represent independently a hydrogen atom or an alkyl group;
R3 represents a hydrogen atom;
R4 and R5 together with the nitrogen atom to which they are attached form a heterocycloalkyl that includes only one nitrogen atom;
R8 represents an aryl group optionally substituted by one or more identical or different groups, wherein said groups are: alkyl, alkoxy; alkylthio; halo; haloalkyl; cyano; nitro; heteroarylthio optionally substituted by one or more identical or different groups, wherein said groups are halo or haloalkyl; aryloxy optionally substituted by one or more nitro groups; or a pharmaceutically acceptable salt of said compound.

7. The compound of claim 6, wherein:
the term heterocycloalkyl denotes a pyrrolidine or a piperidine;
the term aryl in the aryl group and the aryloxy group denotes a phenyl group; and
the heteroarylthio group is the pyridinylthio group;
or a pharmaceutically acceptable salt of said compound.

8. The compound of claim 1, wherein W represents —NR6- or an oxygen atom; or a pharmaceutically acceptable salt of said compound.

9. The compound of claim 8, wherein R1 represents a —C(═O)—NHR8, —C(═S)—NHR8, —C(═S)—NH—C(═O)—R8, —C(═N—CN)—NHR8, —C(═O)—R9 or —SO₂—R10 group; or a pharmaceutically acceptable salt of said compound.

10. The compound of claim 8, wherein R2 represents a hydrogen atom, R4 and R5 together with the nitrogen atom to which they are attached form a heterocycloalkyl containing only carbon, nitrogen and optionally oxygen atoms or a pharmaceutically acceptable salt of said compound.

11. The compound of claim 8, wherein R4 and R5 together with the nitrogen atom to which they are attached form a heterocycloalkyl containing only one nitrogen atom; or a pharmaceutically acceptable salt of said compound.

12. The compound of claim 8, wherein:
R1 represents either a —C(═O)—NHR8 or —C(═S)—NHR8 group;
R2 represents a hydrogen atom;
W represents —NR6- or the oxygen atom;
R6 represents an alkyl group;
R3 represents a hydrogen atom;
R4 and R5 represent independently a hydrogen atom or an alkyl group; or alternatively R4 and R5 together with the nitrogen atom to which they are attached form a heterocycloalkyl including only one nitrogen atom;
R8 represents an aryl group optionally substituted by one or more identical or different groups, wherein said groups are: alkyl, alkoxy; alkylthio; halo; haloalkyl; cyano; nitro; heteroarylthio optionally substituted by one or more identical or different groups, wherein said groups are halo or haloalkyl; aryloxy optionally substituted by one or more nitro groups; or a —SO₂NR15R16 group;
R15 and R16 represent independently a heteroaryl group optionally substituted by one or more identical or different C₁-C₃ alkyls, a C₁-C₃ alkyl group, an aryl group or a hydrogen atom; or alternatively R15 and R16 together may form a heterocycloalkyl including the nitrogen atom;
or a pharmaceutically acceptable salt of said compound.

13. The compound of claim 12, wherein:
the term heterocycloalkyl denotes a pyrrolidine or a piperidine;
the term aryl in the aryl group and the aryloxy group denotes a phenyl group;
the term heteroaryl in the heteroaryl group and the heteroarylthio group represents a pyridine or a pyrimidine;
or a pharmaceutically acceptable salt of said compound.

14. The compound of claim 1, wherein the term aryl in the aryl, aryloxy, arylsulfonyl, arylalkyl, aryloxyalkyl and arylcarbonyl groups represents a phenyl, naphthyl or fluororenyl group;
or a pharmaceutically acceptable salt of said compound.

15. The compound of claim 1, wherein the term heteroaryl in the heteroaryl, heteroarylalkyl and heteroarylthio groups represents a furyl, thienyl isoxazolyl, benzothiadiazolyl, pyridinyl, oxazolyl, pyrazolyl, pyrimidinyl or quinoxalyl group;
or a pharmaceutically acceptable salt of said compound.

16. The compound of claim 1, wherein the term cycloalkyl represents cyclopentyl or cyclohexyl;
or a pharmaceutically acceptable salt of said compound.

17. The compound of claim 1, wherein the term heterocycloalkyl in the heterocycloalkyl group and the heterocycloalkylalkyl group represents a tetrahydrofuryl, azetidinyl, pyrrolidinyl, morpholinyl or piperidyl group;
or a pharmaceutically acceptable salt of said compound.

18. The compound of claim 1, wherein said compound is:
N-{2-[(2,6-dipyrrolidin-1-ylpyrimidin-4-yl)amino]ethyl}-N-methylethane-1,2-diamine;
N-(3-aminopropyl)-N′-(2,6-dipyrrolidin-1-ylpyrimidin-4-yl)-N-methylpropane-1,3-diamine;
N-(2,6-dipyrrolidin-1-ylpyrimidin-4-yl)-N,N′-dimethyl-N′-[3-(methylamino)propyl]propane-1,3-diamine;
N-(2,6-dipyrrolidin-1-ylpyrimidin-4-yl)pentane-1,5-diamine;
N′-(2,6-dipyrrolidin-1-ylpyrimidin-4-yl)-N,N-dimethylpentane-1,5-diamine;
N′-(2,6-dipyrrolidin-1-ylpyrimidin-4-yl)-N,N-diethylpentane-1,5-diamine;
N-[4-chloro-3-(trifluoromethyl)phenyl]-N′-{2-[{2-[(2,6-dipyrrolidin-1-ylpyrimidin-4-yl)amino]ethyl}(methyl)amino]ethyl}urea;
N-benzyl-N′-{2-[{2-[(2,6-dipyrrolidin-1-ylpyrimidin-4-yl)amino]ethyl}(methyl)amino]ethyl}urea;
N-(tert-butyl)-N′-{2-[{2-[(2,6-dipyrrolidin-1-ylpyrimidin-4-yl)amino]ethyl}(methyl)amino]ethyl}urea;
N-{2-[{2-[(2,6-dipyrrolidin-1-ylpyrimidin-4-yl)amino]ethyl}(methyl)amino]ethyl}-N′-2-thienylurea;
N-{2-[{2-[(2,6-dipyrrolidin-1-ylpyrimidin-4-yl)amino]ethyl}(methyl)amino]ethyl}-N′-[(2R)-1,2,3,4-tetrahydronaphthalen-2-yl]urea;
N-cyclopentyl-N′-{2-[{2-[(2,6-dipyrrolidin-1-ylpyrimidin-4-yl)amino]ethyl}(methyl)amino]ethyl}urea;
N-(3,5-dimethylisoxazol-4-yl)-N-{2-[{2-[(2,6-dipyrrolidin-1-ylpyrimidin-4-yl)amino]ethyl}(methyl)amino]ethyl}urea;
N-{2-[{2-[(2,6-dipyrrolidin-1-ylpyrimidin-4-yl)amino]ethyl}(methyl)amino]ethyl}-N′-(2-furylmethyl)urea;
N-2,1,3-benzothiadiazol-4-yl-N′-{2-[{2-[(2,6-dipyrrolidin-1-ylpyrimidin-4-yl)amino]ethyl}(methyl)amino]ethyl}urea;
N-(4-chlorophenyl)-N′-{2-[{2-[(2,6-dipyrrolidin-1-ylpyrimidin-4-yl)amino]ethyl}(methyl)amino]ethyl}urea;
N-(3,4-dichlorophenyl)-N′-{2-[{2-[(2,6-dipyrrolidin-1-ylpyrimidin-4-yl)amino]ethyl}(methyl)amino]ethyl}urea;
N-{2-[{2-[(2,6-dipyrrolidin-1-ylpyrimidin-4-yl)amino]ethyl}(methyl)amino]ethyl}-N′-ethylurea;
N-(4-chlorophenyl)-N′-{5-[(2,6-dipyrrolidin-1-ylpyrimidin-4-yl)amino]pentyl}urea;
N-[4-chloro-3-(trifluoromethyl)phenyl]-N′-(2-{2-[(2,6-dipyrrolidin-1-ylpyrimidin-4-yl)amino]ethoxy}ethyl)urea;
N-[4-chloro-3-(trifluoromethyl)phenyl]-N′-{3-[{3-[(2,6-dipyrrolidin-1-ylpyrimidin-4-yl)amino]propyl}(methyl)amino]propyl}urea;
N-(4-chlorophenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-[4-(trifluoromethoxy)phenyl]thiourea;
N-{2-[{2-[(2,6-dipyrrolidin-1-ylpyrimidin-4-yl)amino]ethyl}(methyl)amino]ethyl}-N′-(4-fluorophenyl)thiourea;
N-{2-[{2-[(2,6-dipyrrolidin-1-ylpyrimidin-4-yl)amino]ethyl}(methyl)amino]ethyl}-N′-[4-(trifluoromethyl)phenyl]thiourea;
N-{2-[{2-[(2,6-dipyrrolidin-1-ylpyrimidin-4-yl)amino]ethyl}(methyl)amino]ethyl}-N′-pyridin-3-ylthiourea;
N-{2-[{2-[(2,6-dipyrrolidin-1-ylpyrimidin-4-yl)amino]ethyl}(methyl)amino]ethyl}-N′-[4-(piperidin-1-ylsulfonyl)phenyl]thiourea;
N-{2-[{2-[(2,6-dipyrrolidin-1-ylpyrimidin-4-yl)amino]ethyl}(methyl)amino]ethyl}-N′-ethylthiourea;
N-{2-[{2-[(2,6-dipyrrolidin-1-ylpyrimidin-4-yl)amino]ethyl}(methyl)amino]ethyl}-N′-(2-furylmethyl)thiourea;
N-{2-[{2-[(2,6-dipyrrolidin-1-ylpyrimidin-4-yl)amino]ethyl}(methyl)amino]ethyl}-N′-(6-phenoxypyridin-3-yl)thiourea;
N-{2-[{2-[(2,6-dipyrrolidin-1-ylpyrimidin-4-yl)amino]ethyl}(methyl)amino]ethyl}-N′-(tetrahydrofuran-2-ylmethyl)thiourea;
N-{2-[{2-[(2,6-dipyrrolidin-1-ylpyrimidin-4-yl)amino]ethyl}(methyl)amino]ethyl}-N′-(6-morpholin-4-ylpyridin-3-yl)thiourea;
N-{2-[{2-[(2,6-dipyrrolidin-1-ylpyrimidin-4-yl)amino]ethyl}(methyl)amino]ethyl}-N′-[4-(1,3-oxazol-5-yl)phenyl]thiourea;
N-{2-[{2-[(2,6-dipyrrolidin-1-ylpyrimidin-4-yl)amino]ethyl}(methyl)amino]ethyl}-N′-(pentafluorophenyl)thiourea;
N-{2-[{2-[(2,6-dipyrrolidin-1-ylpyrimidin-4-yl)amino]ethyl}(methyl)amino]ethyl}-N′-(4-methoxyphenyl)thiourea;
N-{2-[{2-[(2,6-dipyrrolidin-1-ylpyrimidin-4-yl)amino]ethyl}(methyl)amino]ethyl}(4-phenoxyphenyl)thiourea;
N-{2-[{2-[(2,6-dipyrrolidin-1-ylpyrimidin-4-yl)amino]ethyl}(methyl)amino]ethyl}-N′-1-naphthylthiourea;
N-{2-[{2-[(2,6-dipyrrolidin-1-ylpyrimidin-4-yl)amino]ethyl}(methyl)amino]ethyl}-N′-(3,4,5-trimethoxyphenyl)thiourea;
N-{2-[{2-[(2,6-dipyrrolidin-1-ylpyrimidin-4-yl)amino]ethyl}(methyl)amino]ethyl}-N′-(3-fluorophenyl)thiourea;
N-(2,4-difluorophenyl)-N′-{2-[{2-[(2,6-dipyrrolidin-1-ylpyrimidin-4-yl)amino]ethyl}(methyl)amino]ethyl}thiourea;
N-(3,5-difluorophenyl)-N-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-(2-fluorophenyl)thiourea;
N-{2-[{2-[(2,6-dipyrrolidin-1-ylpyrimidin-4-yl)amino]ethyl}(methyl)amino]ethyl}-N′-(4-nitrophenyl)thiourea;
N-(4-tert-butylphenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-[4-(4-nitrophenoxy)phenyl]thiourea;
N-{2-[{2-[(2,6-dipyrrolidin-1-ylpyrimidin-4-yl)amino]ethyl}(methyl)amino]ethyl}-N′-(4-fluorobenzyl)thiourea;
N-[2-(2,4-difluorophenoxy)pyridin-3-yl]-N′-{2-{[2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-[4-(1H-pyrazol-1-yl)phenyl]thiourea;
N-{2-[{2-[(2,6-dipyrrolidin-1-ylpyrimidin-4-yl)amino]ethyl}(methyl)amino]ethyl}-N′-(3-nitrophenyl)thiourea;
N-(4,6-dimethylpyrimidin-2-yl)-4-{[({2-[{2-[(2,6-dipyrrolidin-1-ylpyrimidin-4-yl)amino]ethyl}(methyl)amino]ethyl}amino)carbonothioyl]amino}benzene-sulfonamide;
N-{2-[{2-[(2,6-dipyrrolidin-1-ylpyrimidin-4-yl)amino]ethyl}(methyl)amino]ethyl}-N′-[4-(methylthio)phenyl]thiourea;
N-(4-{[3-chloro-5-(trifluoromethyl)pyridin-2-yl]thio}phenyl)-N′-{2-[{2-[(2,6-dipyrrolidin-1-ylpyrimidin-4-yl)amino]ethyl}(methyl)amino]ethyl}thiourea;
N-(6-chloropyridin-3-yl)-N-{2-[{2-[(2,6-dipyrrolidin-1-ylpyrimidin-4-yl)amino]ethyl}(methyl)amino]ethyl}thiourea;
N-[4-chloro-3-(trifluoromethyl)phenyl]-N′-{2-[{2-[(2,6-dipyrrolidin-1-ylpyrimidin-4-yl)amino]ethyl}(methyl)amino]ethyl}thiourea;
N-(3,4-dichlorophenyl)-N′-{2-[{2-[(2,6-dipyrrolidin-1-ylpyrimidin-4-yl)amino]ethyl}(methyl)amino]ethyl}thiourea;
N-(4-chloro-3-fluorophenyl)-N′-{2-[{2-[(2,6-dipyrrolidin-1-ylpyrimidin-4-yl)amino]ethyl}(methyl)amino]ethyl}thiourea;
N-[4-chloro-3-(trifluoromethyl)phenyl]-N′-{5-[(2,6-dipyrrolidin-1-ylpyrimidin-4-yl)amino]pentyl}thiourea;
N-(4-chlorophenyl)-N′-{5-[(2,6-dipyrrolidin-1-ylpyrimidin-4-yl)amino]pentyl}thiourea;
4-{[({2-[{2-[(2,6-dipyrrolidin-1-ylpyrimidin-4-yl)amino]ethyl}(methyl)amino]ethyl}amino)carbonothioyl]amino}-N-methylbenzenesulfonamide;
N-{4-[(4-bromophenyl)sulfonyl]phenyl}-N′-{2-[{2-[(2,6-dipyrrolidin-1-ylpyrimidin-4-yl)amino]ethyl}(methyl)amino]ethyl}thiourea;
4-{[({2-[{2-[(2,6-dipyrrolidin-1-ylpyrimidin-4-yl)amino]ethyl}(methyl)amino]ethyl}amino)carbonothioyl]amino}benzenesulfonamide;
4-{[({2-[{2-[(2,6-dipyrrolidin-1-ylpyrimidin-4-yl)amino]ethyl}(methyl)amino]ethyl}amino)carbonothioyl]amino}-N-phenylbenzenesulfonamide;
N˜6˜-{2-[(2-aminoethyl)(methyl)amino]ethyl}-N˜2˜,N˜4˜-bis[2-(dimethylamino)ethyl]-N˜2˜,N˜4˜-dimethylpyrimidine-2,4,6-triamine
N-{2-[[2-({2,6-bis[[2-(dimethylamino)ethyl](methyl)amino]pyrimidin-4-yl}amino)ethyl](methyl)amino]ethyl}-N′-(4-chlorophenyl)thiourea;
N-{2-[(2,6-dimorpholin-4-ylpyrimidin-4-yl)amino]ethyl}-N-methylethane-1,2-diamine
N-(4-chlorophenyl)-M-{2-[{2-[(2,6-dimorpholin-4-ylpyrimidin-4-yl)amino]ethyl}(methyl)amino]ethyl}thiourea;
N-(3,4-difluorophenyl)-N′-{2-[{2-[(2,6-dipyrrolidin-1-ylpyrimidin-4-yl)amino]ethyl}(methyl)amino]ethyl}thiourea;
N-{2-[(2,6-dipiperidin-1-ylpyrimidin-4-yl)amino]ethyl}-N-methylethane-1,2-diamine;
N-(4-chlorophenyl)-N′-{2-[{2-[(2,6-dipiperidin-1-ylpyrimidin-4-yl)amino]ethyl}(methyl)amino]ethyl}thiourea;
N˜6˜-{2-[(2-aminoethyl)(methyl)amino]ethyl}-N˜2˜,N˜2˜,N˜4˜,N˜4˜-tetraethylpyrimidine-2,4,6-triamine;
N-{2-[(2-{[2,6-bis(diethylamino)pyrimidin-4-yl]amino}ethyl)(methyl)amino]ethyl}-N′-(4-chlorophenyl)thiourea;
N˜6˜-{2-[(2-aminoethyl)(methyl)amino]ethyl}-N˜2˜,N˜2˜,N˜4˜,N˜4˜-tetramethylpyrimidine-2,4,6-triamine;
N-{2-[(2-{[2,6-bis(dimethylamino)pyrimidin-4-yl]amino}ethyl)(methyl)amino]ethyl}-N′-(4-chlorophenyl)thiourea;
N-{2-[(2,6-diazetidin-1-ylpyrimidin-4-yl)amino]ethyl}-N-methylethane-1,2-diamine;
N-(4-chlorophenyl)-N′-{2-[{2-[(2,6-diazetidin-1-ylpyrimidin-4-yl)amino]ethyl}(methyl)amino]ethyl}thiourea;
N-[4-(dimethylamino)phenyl]-N′-{2-[{2-[(2,6-dipyrrolidin-1-ylpyrimidin-4-yl)amino]ethyl}(methyl)amino]ethyl}thiourea;
N-(4-cyanophenyl)-N-{2-[{2-[(2,6-dipyrrolidin-1-ylpyrimidin-4-yl)amino]ethyl}(methyl)amino]ethyl}thiourea;
N˜4˜-{2-[(2-aminoethyl)(methyl)amino]ethyl}-N˜2˜,N˜6˜-diethylpyrimidine-2,4,6-triamine
N-{2-[(2-{[2,6-bis(ethylamino)pyrimidin-4-yl]amino}ethyl)(methyl)amino]ethyl}-N′-(4-chlorophenyl)thiourea;
N-[({2-[{2-[(2,6-dipyrrolidin-1-ylpyrimidin-4-yl)amino]ethyl}(methyl)amino]ethyl}amino)carbonothioyl]-4-methoxybenzamide;
N-(4-chlorophenyl)-N″-cyano-N′-{2-[{2-[(2,6-dipyrrolidin-1-ylpyrimidin-4-yl)amino]ethyl}(methyl)amino]ethyl}guanidine;
N-{2-[{2-[(2,6-dipyrrolidin-1-ylpyrimidin-4-yl)amino]ethyl}(methyl)amino]ethyl}quinoxaline-2-carboxamide
4-benzoyl-N-{2-[{2-[(2,6-dipyrrolidin-1-ylpyrimidin-4-yl)amino]ethyl}(methyl)amino]ethyl}benzamide;
N-{2-[{2-[(2,6-dipyrrolidin-1-ylpyrimidin-4-yl)amino]ethyl}(methyl)amino]ethyl}-2-phenoxypropanamide;
N-{2-[{2-[(2,6-dipyrrolidin-1-ylpyrimidin-4-yl)amino]ethyl}(methyl)amino]ethyl}-9-oxo-9H-fluorene-4-carboxamide;
N-{2-[{2-[(2,6-dipyrrolidin-1-ylpyrimidin-4-yl)amino]ethyl}(methyl)amino]ethyl}-1-[4-(trifluoromethyl)pyrimidin-2-yl]piperidine-4-carboxamide;
N-{2-[{2-[(2,6-dipyrrolidin-1-ylpyrimidin-4-yl)amino]ethyl}(methyl)amino]ethyl}-4-nitrobenzenesulfonamide;
N-{2-[{2-[(2,6-dipyrrolidin-1-ylpyrimidin-4-yl)amino]ethyl}(methyl)amino]ethyl}-3-(trifluoromethyl)benzenesulfonamide; or
N-{2-[{2-[(2,6-dipyrrolidin-1-ylpyrimidin-4-yl)amino]ethyl}(methyl)amino]ethyl}-4-(trifluoromethyl)benzenesulfonamide;
or a pharmaceutically acceptable salt thereof of said compound.

19. The compound of claim 1, wherein said compound is:
N-[4-chloro-3-(trifluoromethyl)phenyl]-N′-(2-{2-[(2,6-dipyrrolidin-1-ylpyrimidin-4-yl)amino]ethoxy}ethyl)urea;
N-(4-chlorophenyl)-N′-{5-[(2,6-dipyrrolidin-1-ylpyrimidin-4-yl)amino]pentyl}urea;
N-[4-chloro-3-(trifluoromethyl)phenyl]-N′-(2-{2-[(2,6-dipyrrolidin-1-ylpyrimidin-4-yl)amino]ethoxy}ethyl)urea;
N-[4-chloro-3-(trifluoromethyl)phenyl]-N′-{3-[{3-[(2,6-dipyrrolidin-1-ylpyrimidin-4-yl)amino]propyl}(methyl)amino]propyl}urea;
N-(4-chlorophenyl)-N′-2-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-[4-(piperidin-1-yl sulfonyl)phenyl]thiourea;
N-{2-[{2-[(2,6-dipyrrolidin-1-ylpyrimidin-4-yl)amino]ethyl}(methyl)amino]ethyl}-N′-(pentafluorophenyl)thiourea;
N-{2-[{2-[(2,6-dipyrrolidin-1-ylpyrimidin-4-yl)amino]ethyl}(methyl)amino]ethyl}-N′-(4-methoxyphenyl)thiourea;
N-{2-[{2-[(2,6-dipyrrolidin-1-ylpyrimidin-4-yl)amino]ethyl}(methyl)amino]ethyl}-N′-(4-phenoxyphenyl)thiourea;
N-{2-[{2-[(2,6-dipyrrolidin-1-ylpyrimidin-4-yl)amino]ethyl}(methyl)amino]ethyl}-N′-(3-fluorophenyl)thiourea;
N-(2,4-difluorophenyl)-N′-{2-[{2-[(2,6-dipyrrolidin-1-ylpyrimidin-4-yl)amino]ethyl}(methyl)amino]ethyl}thiourea;
N-(3,5-difluorophenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}-N′-(4-nitrophenyl)thiourea;
N-(4-tert-butylphenyl)-N′-{2-[{2-[(2,6-dipyrrolidin-1-ylpyrimidin-4-yl)amino]ethyl}(methyl)amino]ethyl}thiourea;
N-{2-[{2-[(2,6-dipyrrolidin-1-ylpyrimidin-4-yl)amino]ethyl}(methyl)amino]ethyl}N-(3-nitrophenyl)thiourea;
N-(4,6-dimethylpyrimidin-2-yl)-4-{[({2-[{2-[(2,6-dipyrrolidin-1-ylpyrimidin-4-yl)amino]ethyl}(methyl)amino]ethyl}amino)carbonothioyl]amino}benzenesulfonamide;
N-{2-{2-[(2,6-dipyrrolidin-1-ylpyrimidin-4-yl)amino]ethyl}(methyl)amino)ethyl}-N′-[4-(methylthio)phenyl]thiourea;
N-(4-{[3-chloro-5-(trifluoromethyl)pyridin-2-yl]thio}phenyl)-N′-{2-{[2-[(2,6-dipyrrolidin-1-ylpyrimidin-4-yl)amino]ethyl}(methyl)amino]ethyl}thiourea;
N-[4-chloro-3-(trifluoromethyl)phenyl]-N′-{2-[{2-[(2,6-dipyrrolidin-1-ylpyrimidin-4-yl)amino]ethyl}(methyl)amino]ethyl}thiourea;
N-(3,4-dichlorophenyl)-N′-{2-[{2-[(2,6-dipyrrolidin-1-ylpyrimidin-4-yl)amino]ethyl}(methyl)amino]ethyl}thiourea;
N-(4-chloro-3-fluorophenyl)-N′-{2-[{2-[(2,6-dipyrrolidin-1-ylpyrimidin-4-yl)amino]ethyl}(methyl)amino]ethyl}thiourea
N-[4-chloro-3-(trifluoromethyl)phenyl]-N′-{5-[(2,6-dipyrrolidin-1-ylpyrimidin-4-yl)amino]pentyl}thiourea;
N-(4-chlorophenyl)-N′-{5-[(2,6-dipyrrolidin-1-ylpyrimidin-4-yl)amino]pentyl}thiourea;
4-{[({2-[{2-[(2,6-dipyrrolidin-1-ylpyrimidin-4-yl)amino]ethyl}(methyl)amino]ethyl}amino)carbonothioyl]amino}-N-methylbenzenesulfonamide;
N-{4-[(4-bromophenyl)sulfonyl]phenyl}-N′-{2-[{2-[(2,6-dipyrrolidin-1-ylpyrimidin-4-yl)amino]ethyl}(methyl)amino]ethyl}thiourea
4-{[({2-[{2-[(2,6-dipyrrolidin-1-ylpyrimidin-4-yl)amino]ethyl}(methyl)amino]ethyl}amino)carbonothioyl]amino}benzenesulfonamide;
4-{[({2-[{2-[(2,6-dipyrrolidin-1-ylpyrimidin-4-yl)amino]ethyl}(methyl)amino]ethyl}amino)carbonothioyl]amino}-N-phenylbenzenesulfonamide;
N-(3,4-difluorophenyl)-N′-{2-{[2-[(2,6-dipyrrolidin-1-ylpyrimidin-4-yl)amino]ethyl}(methyl)amino]ethyl}thiourea;
N-(4-chlorophenyl)-N′-{2-[{2-[(2,6-dipiperidin-1-ylpyrimidin-4-yl)amino]ethyl}(methyl)amino]ethyl}thiourea;
N-{2-[{2-[2,6-bis(diethylamino)pyrimidin-4-yl]amino}ethyl)(methyl)amino]ethyl}-N′-(4-chlorophenyl)thiourea;
N-(4-cyanophenyl)-N′-{2-[{2-[(2,6-dipyrrolidin-1-ylpyrimidin-4-yl)amino]ethyl}(methyl)amino]ethyl}thiourea;
or a pharmaceutically acceptable salt of said compound.

20. A method for preparing a compound having the general formula (I) 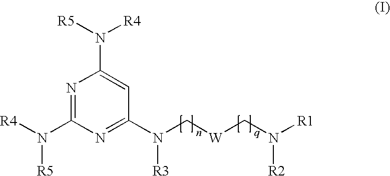
in a racemic form, an enantiomeric form or any combination thereof, wherein:
R1 represents a hydrogen atom, an alkyl group, a —C(═O)—NHR8, —C(═S)—NHR8, —C(═S)—NH—C(═O)—R8, —C(═N—CN)—NHR8, —C(═O)—R9 or —SO₂—R10 group;
R2 represents a hydrogen atom, a C₁-C₃ linear or branched alkyl group;
W represents —NR6-, —CR6R7-, an oxygen atom or a sulfur atom;
R6 and R7 represent independently a hydrogen atom or an alkyl group;
n and q are integers from 2 to 6 inclusive;
R3 represents a hydrogen atom or an alkyl group;
R4 and R5 represent independently a hydrogen atom, an alkyl group, an aminoalkyl, alkylaminoalkyl or dialkylaminoalkyl group; or alternatively R4 and R5 together with the nitrogen atom to which they are attached form a heterocycloalkyl;
R8 represents a hydrogen atom or one of the following groups:
alkyl,
cycloalkyl,
heterocycloalkylalkyl,
heteroaryl optionally substituted by one or more identical or different groups, wherein said groups are: alkyl, heterocycloalkyl, halo or aryloxy optionally substituted by one or more identical or different halogens,
heteroarylalkyl,
aryl optionally substituted by one or more identical or different groups, wherein said groups are: alkyl; alkoxy; alkylthio; dialkylamino; halo; haloalkyl; haloalkyloxy; cyano; nitro; heteroaryl; heteroarylthio optionally substituted by one or more identical or different groups, wherein said groups are: halo or haloalkyl; aryloxy optionally substituted by one or more nitro groups; arylsulfonyl optionally substituted by one or more identical or different halogens: or a —SO₂NR15R16 group;
arylalkyl optionally substituted by one or more identical or different halogens, or;
a group having the formula: 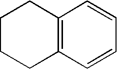
R9 represents one of the following groups:
aryl optionally substituted by one or more arylcarbonyl groups,
aryloxyalkyl optionally substituted by a C₁-C₃ alkyl group,
heteroaryl;
heterocycloalkyl optionally substituted by a heteroaryl, itself optionally substituted by a haloalkyl group;
R10 represents an aryl group optionally substituted by one or more identical or different groups, wherein said groups are: haloalkyl or nitro;
R15 and R16 may together form a heterocycloalkyl group including the nitrogen atom, or R15 and R16 independently represent a heteroaryl group optionally substituted by one or more identical or different C₁-C₃ alkyls is a C₁-C₃ alkyl group, an aryl group or a hydrogen atom;
or a pharmaceutically acceptable salt of said compound;
said method comprising the steps of:
A) reacting a compound of general formula (II) 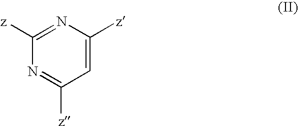
wherein z, z′ and z″ represent a halogen atom,
with an amine of general formula R5R4NH, at a temperature between −5° C. and 5° C., in an inert solvent to form the compound of general formula (IV) 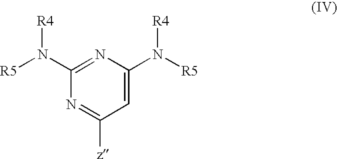
wherein z″ represents a halogen atom;
B) reacting the resulting compound of general formula (IV) with the diamine of general formula R3HN—(CH₂)ₙ—W—(CH₂)_{q}—NR1R2 wherein R1 and R2 represent independently a hydrogen atom or an alkyl group,
by heating to a temperature between 150° C. and 250° C., to form the compound of general formula (I) wherein R1 and R2 represent independently a hydrogen atom or an alkyl group; and
C) optionally further reacting the resulting compound wherein R1 is a hydrogen atom with:
either an isocyanate or isothiocyanate compound of general formula R8NCY, wherein Y represents a sulfur or oxygen atom, to a temperature between 10° C. and 30° C. in a solvent selected from dichloromethane, 1,2-dichloromethane or dimethylformamide to give the compound of general formula (I) wherein R1 represents —C(═Y)—NHR8 (compound Ib); 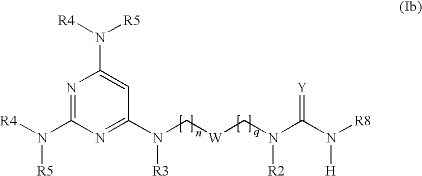
or with
a carboxy-isothiocyanate derivative of general formula R8C(O)NCS, at a temperature between 10° C. and 30° C. in a solvent selected from dichloromethane, 1,2-dichloromethane or dimethylformamide to give the compound of general formula (I) wherein R1 represents —C(═S)—NHC(═O)R8 (compound Ic); 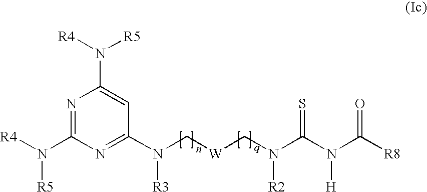
or with
a cyanoethylene derivative in its salt form, of general formula (IX), 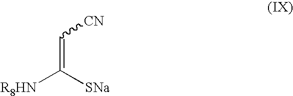
at the reflux temperature of a polar solvent, to give the compound of general formula (I) wherein R1 represents —C(═N—CN)—NHR8 (compound Id), 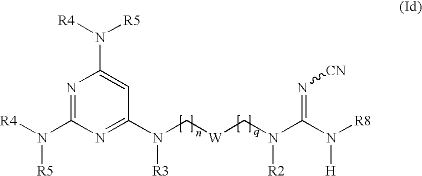
or with
of an acyl halide compound of general formula R9C(O)_{z}′, wherein z′ represents a halogen atom, in the presence of a tertiary amine at a temperature between 10° C. and 30° C. in an inert solvent, or alternatively a compound of general formula R9CO₂H, in the presence of a peptide coupling agent, at a temperature between 10° C. and 30° C. (preferably 20° C.) in an inert solvent to give the compound of general formula (I) wherein R1 represents —C(═O)—R9 (compound Ie); 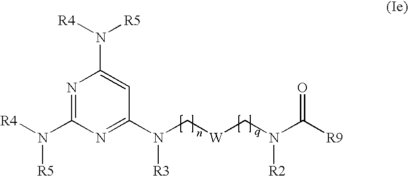
or with
an arylsulfonyl halide compound of formula R10SO₂z′, wherein z′ represents a halogen atom, in the presence of a tertiary amine in a solvent selected from dichloromethane, 1,2-dichloromethane or dimethylformamide at a temperature between 10° C. and 30° C. to give the compound of general formula (I) wherein R1 represents —SO₂—R10 (compound If)

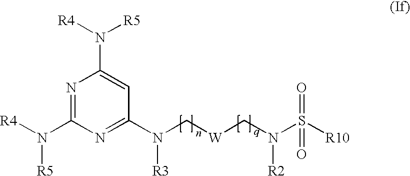

21. A compound, wherein said compound is:
2,4-diazetidin-1-yl-6-chloropyrimidine; or
6-chloro-N,N′-bis[2-(dimethylamino)ethyl]-N,N′-dimethylpyrimidine-2,4-diamine.

22. A pharmaceutical composition comprising the compound of general formula (I) of claim 1, or a pharmaceutically acceptable salt of such a compound, and at least one pharmaceutically acceptable excipient.

23. A medicament comprising the compound of general formula (I) of claim 1, or a pharmaceutically acceptable salt of said compound.

24. A method for treating or preventing disease comprising the step of administering to a patient in need thereof a therapeutically effective amount of a compound of general formula (I) of claim 1, or a pharmaceutically acceptable salt thereof, to treat or prevent: cancers, cancerous proliferative diseases, noncancerous proliferative diseases, neurodegenerative diseases, parasitic diseases, viral infections, spontaneous alopecia, alopecia induced by exogenous products, radiation-induced alopecia, autoimmune diseases, graft rejection, inflammatory diseases or allergies.

25. The method of claim 24, wherein a therapeutically effective amount of said compound is administered to a patient in need thereof to treat or prevent cancer.

26. The method of claim 25, wherein said cancer to be treated or prevented is: cancer of the colon, rectum, stomach, lung, pancreas, kidney, testicle, breast, uterus, ovary, prostate, skin, bone, spinal cord, neck, tongue, or head, or sarcomas, carcinomas, fibroadenomas, neuroblastomas, leukaemias, or melanomas.
